SEQUENCE LISTING (13 sequences)

SEQ ID NO: 1 - protein, 1180 aa, Arabidopsis thaliana
MDAERSSLCNFFVNFLMEENYLLTAFELLHELLDDGRDAQAIRLKEFFSDPSRFPPDQISRYNSIRVADPQSLLEEKEALAEKLAISEYEFRLAQEDIARLKTEGQKKSVPSIDKSEEMDSDEFGGNRPEIQRKKKDFSFTDIGPLKNNERQDLNCAVKEYLLLAGYRLTAMTFYEEVTDQNLDVWQDSPAHVPDALRYYYYQYLSSTSEAAEEKIAMLQENESLKKEIERLSKEKDGLLKSKENFEEQIGAFNKSTESLQKDLRDREKQVQSLKQSSEHQRRNLNDCRAEITSLKMHIEGSRAGQYVSLNEGDPVKLQSKEVEEQISTLSEEVVNPTVEKDGGLISKVSISAEKGHIQTEDDMVVEEVKNIIADQREVAGEAGNISYANNGTLENQKEVSNYLLSPSNGNFSPRDLGSILKVDPGIGRDSNSKSDNANGEAASEEMGLGTIQILADALPNIVPYVLINHREELLPLMMCAIERHPVSSTRDSLTHTLFNLIKRPDEQQRRIIMDACVSLSRNVGEMRTETELLPQCWEQINHTYEERRLLVAQSCGELAEYVRPEIRDSLILSIVQQLIEDSATVVREAAAHNLALLLPLFPNTDKYFKVEEMMFQLICDPSGLVVETTLKELLPAVIKWGNRLDHILRGLLSHTLSSAQHCPPLSGVEGSLESHLRVLGERERWNIDVLLRMLMELLPAIHQKAMTTCPFSSISKSEESAFSVSLLEIYAEGRSEWPMFEWMHVDCFANLLQLACMLPQKEDHLRNRITKFLLAVSERFGSSYLTHIELPVFLVAAGDDEADLRFLPSAIHPRIKGLKPRTAVANRLATLCILPLLLAGVLGAPSKREELTIFLRQLLVESKTKENQSSKHNNEVLDAVRFLCTFEVHHNMIFGILWEMVVDSTAELKINAAKLLKTIVPYIDAKVASANVLPALITLGSDQNLNVKYASIDAFGSVAQHFKVDMIVDKILVQMDAFMEDGSHEAIIAVIRALLVAIPHTTERLRDYLLSKILQLSASPSSSTDVNRRRERANAFCEAIRALDATDLSQTSVKEYLLPAIQNLLKDPDALDPAHKEALEIIMKERSGGTFEAISKAMGAHLGIASSVTSLFGEGGLLGKKEATESTAPGSPTGQGPESPKVVAAASEDTRFRRIMRGNFTEMLRSKAKTQDETQPQNH

SEQ ID NO: 2 - protein, 1184 aa, Vitis vinifera
MDVVERASLCNCVVNFLLEEKYLLSAFELLHELLEDGREAQAIRLKEFFSDPSHFPPDQISRFNSLRVADPQSLLEEKEALEEKLAISGYELRLAQEDILKLKTELEKKADFLPNSSKSNSDVSVDHGQDIQRQKRDASYSDLGPLKDNERRDLNCAVKEYLLLAGYRLTAMTFYEEVIDQNLDVWQNTPACVPDALRHYYYQYLSSTAEAAEEKIAMLRENESLLKANENLNHEKECLLKNKDLADGQIKALTKSSEALQKDLKDRENLVQVLKQSLEHQRKDLNDCRAEITSLKMHIEGYRSGRSWATSDVDDVQSSLERYKEEIKSLQMEMESLKAKNSIATDALDSSNCGKESIQGEENVVEIHEDKTVISHQVDTTSGVLENQDAPLLACQTSDDNMKKPEEVAQELLISSSSENGTAGNVVNAPKQNGEPPPEESEVLKSDNIGGKIVSEKTGLGTIQILSDALPKIVPYVLINHREELLPLIMCAIERHPDSSTRDSLTHTLFNLIKRPDEQQRRIIMDACVNLAKNVGEMRTETELLPQCWEQINHIYEERRLLVAQSCGELAEFVRPEIRDSLILSIVQQLIEDSGTVVRDAAAHNLALLLPLFPNMDKYFKVEELMFQLVCDPSGVVVETTLKELVPAVINWGNKLDHILRILLSHILGSSQRCPPLSGVEGSVESHLHVLGERERWNVDVLLRMLTELLPFVHQKAIETCPFPTVSESMGTLFSTSLLELYAGGHIEWPAFEWMHIDCFPSLIQLACLLPQKEDNLRNRITKFLLAVSERFGDSYLTHIMLPVFLVAIGDNADLTFFPSTIHSVIKGLRPKTAIAERLATMCVLPLLLAGVLGAPCKHEQLVEYLRNLLVQGTVKESQPTKRNAEIVDAVRFLCTFEEHHGMIFNILWEMVVSSNIEMKISAANLLKVIVPYIDAKVASTHVLPALVTLGSDQNLNVKYASIDAFGAVAQHFKNDMIVDKIRVQMDAFLEDGSHEATIAVVRALVVAIPHTTDKLRDYLLSKIFQFTTMPSPTSDVMRRRERANAFCESIRALDATDLPATSVRELLLPAIQNLLKDLDALDPAHKEALEIILKERSGGTLEAISKVMGAHLGIASSVTSLFGEGGLLGKKDSGDPPPEPVESPRAVPPPPAEDTRFMRIMRGNFTDMLRSKAKNQEDTSTGQ

SEQ ID NO: 3 - protein, 1184 aa, Gossypium hirsutum
MMDVERSSLCNCVVNFLLEENYLLTAFELLHELLDDGRDAQAIRLKEFFSDPSHFPPDQISRYNSLRVLDPQSLLEEKEAIEEKLALCEYELRLAQEDIVKLKTELQRKEDLTQDKSSEPSVSNHVNHAPEIHRQKRDAPFSDLGPLKANERKDLNCAVKEYLLVAGYRLTAMTFYEEVTDQNLDVWEKSPASVPDALRHYYYQYLSSTSEAAEEKISMIRENDSLKEANESLNHEKLCLMKNKELAEGQINALTKSLEATQKDLKDKENLTQDLKQALENQRKELNDCRAEITSLKMHIEGSSSLQNPVAANLDSTQSQAIESYKEEIKSLQMEIERLRAEKTNISDLVDSSCGDKESIQTEEKVVEMDENRTQISHHIEPAEVVDSNTLVMPVQTFDNSTPKPEENLPESIMNPSNSTDGFPDGRNLSQQEEKPPSEDSRLHLNSENLGSEPVPENMGLATIQILADALPKIVPYVLINHREELLPLIMCAIERHPDSSTRDSLTHTLFNLIKRPDEQQRRIIMDACVNLAKNVGEMRTETELLPQCWEQINHTYEERRLLVAQSCGELAEFVRPEIRDSLILSIVQQLIEDPATVVREAAAHNLALLLPLFPLMDKYFKVEELMFQLACDPSGVVVETTLKELLPAIINWGNKLDHILRVLLSHILGSAQRCPPLSGVEGSAESHLRVLGEQERWNLDVSLRMLAKLLPFIHQKAIETCPFSSVSDSNRTLLSSSILELYAGGHVEWPAFEWVHVDCFSGLLQLVCLLPQKEDNLRIRMTKFLLAVSECFGDSYLIHIMLPVFLIAIGDDADLTFFPPNIHSRIKGLRPRTAVAERLAILGVLPLLLAGVLGSPGKRQQLADYLRKLLLEGATKESRSTTHNMDIVNAVRFLCTFEEHHGMIFNILWEMVVSSNIEMKISAANILKVIVPYIDAKVASTHVLPALITLGSDQNLNVKYASIDAFGAVAQHFKNDMIVDKIRVQMDAFLEDGSLEATIAVVRSLVVAVPHTTERLRDYLLSKIFQLTNTPVSSTDVMRRRQRANAFCEAIRALDATDLSANSIRDFLLPTIQNLLKDPDALDPAHKEALEIILKERSGGTFEALSKVMGAHLGIASSVTSFFGEGGLLGKKEITESPTEPVEALQSAASPAAAEDTRFMRIMRVTDMLRGKGKNPEETHQNQ

SEQ ID NO: 4 - protein, 1184 aa, Glycine max
MDVERSSLCNCVVNFLLEENYLLTAFELLHELLDDGRDDQAIRLKQFFSDPNLFPPDLISRLNSLRVADPQTLLEEKEAAEEKLAISDYELRLAQEDISKLKSELQKKAENHNELKAEKISGDVSVNDGQQIQQKKNSSFTDLGPLKETERRDLNCAVKEYLLIAGYRLTAMTFYEEVTDQNLDSWHNTPASVPDALRHYYYQYLSSTSEAAEEKFSLLRENETLLNANKRLNQEKENLLKNKDMADAQIVTLTKSLDAMQKDLKDKENLVQVLKQSLEHQRKELNDCRAEITSLKVHIEGSHLGNNLVISDVNNVQSESLEKYKEEMKKLQMENEWLKEKNIRSPEPGNFVGSEKENLQINDKVIEIHEDQGAISDPIDVALGAVHNEDAQSPVVQTLAQYADKHEDTLPELFNPANTNNAFKNIKNVSEQNVGQQAEDSSLLVKSDSVNDGAISERTGLGTIQILADALPKIVPYVLINHREELLPLIMCAIERHPDSSTRDSLTHTLFNLIKRPDEQQRRIIMDACVNLAKNVGEMRTETELLPQCWEQISHMYEERRLLVAQSCGELADFVRLEIRNSLILSIVQQLIEDSASVVREAAARNLAMLLPLFPNMDKYFKVEDMMFQLVCDPSGVVVETTLKELVPAVIKWGNKLDHVLRVLLSHIVNSALRCPPLSGVEGSIESNLRVLGERERWNIDILLRMLAELLSWVHQKVIETCPFSSTTETTQAVLSTALLELYARGQVEWGAFEWMHVECFPNLIQLACLLPQKEDNLRSRISKFLLSVSESFGDSYVTCIMLPVFLIAVGDDADLTFFPTSIHSRIKGLRPRSAVADRLSTMCVLPLLLAGVLSAPGKHEQLAEYLRKLLLEENSMQNQSTKHTPEIINAIRFICIYEENHGMIFNILWEMVVSSNASMKINAAKLLKVIVPHIDAKVASTHVLPALVTLGSDQNLTVKYGSIDAFGAVAQHFKNEMIVDKIRVQMDAFLEDGSHEATIAVIRALVVAVPHTTERLREYLLSKISQLTAMPNSSSDLMRRRERANAFCEAIRALDATDLPANSVRDLFLPAIQNLLKDLDALDPAHKEALEIIMKERSGGTFESFSKVMGAHIGLPSSVTSFFGESGLLGKKETTEPPSEATVSPKAAAPSPAEDTRFKRIMLGNFSEMLRGKAKAPEEGQNQ

SEQ ID NO: 5 - protein, 1209 aa, Solanum lycopersicum
MDVEKSSLCNCVVNFLLEENYLLSAFELLHELLDDGRDDQAIRLKQFFSDPSQFPPDQISRFNSIRVADPQSLLEEKEALEEKLALCEYEFRLSQEDIVQLKSELQKKSQTSPCPISDLKIDPSENHGTDSQQQKREGSFSDLGPLKDNERKDLNYAVKEYLLLAGYRLTAMTLLEEVTDQDLDVQQNSSACIPDALRHYYYQYLSSTSEAAEEKIAMLRENESLVKENDKLKHEKQSLLKSKDMADAQVTVLAKSLEALQKEMKDKEILVQSLKQSLESQRQELNECRAEITSLKMHIEGARSARNFIASDFEGVDLPSTDSYKEEIKVLQNEIRRLKLARNSLNSESLENINEETRNTCPENEVEKSSDHNVFDDSAGVSSGDLGAAGSQLSMTQTSDSQLLMSQTSADTITEPERVVEVSHDNCVGDKVENVLKHNGELPSEAKGLILKPDNLLVESNAQKISLGTIQILSDALPKIVPYVLINHREELLPLIMCAIERHPDSSTRDSLTHTLFNLIKRPDEEQRRIIMDACVTLARNVGEMRTETELLPQCWEQINHMYEERRLLVAQSCGELAEFVRPEIRDSLILSIVQQLIEDSATVVREASAHNLALLLPLFPSRDKYFKVEEMMFQLVCDPSGVVVETTIKELVPALVNWGKELDHLLQVLLSHALGSAQRCQPLSGVEGSIESHLRALGERERWNIDVLMRLLSELFPFVRKKAIDTCPFPLVSDDERLVFSTSVLEQYAGGKMDWPSFEWLHIDCFSALIELASLLPQKEDNLRNRITRFLLAVSDLLGEPYLTHIMLPVFLVAVGDDGDLSYFPATCQSRIRGLKPKTAVAERLATIGVLPLLLAGVLGSPRKHELLTEYLRNLLIQTSGQESQTVKREIFFSVRFLCTFDEHHNMIFNILWEMVVSSEINMKATAANLFKVIVPCIDAKVASTHVLPALVTLGSDQNLNVKYASIDAFGAVAQQYKNDAIVDKIRVQMDAFLEDGSHEATLAVVRALVMAVPHTTEGLRDYILNLLAFMHCLNIHLLSKIFLLTATPPPSSDMMRRRERANTFCESIRALDATDLSASSVRDFLLPAIQNLLKDADSLDPAHKEALEIVMRERSGGTFDTISKVMGAHLGIASSVSSFFGEGGLLGKREAGDPTSPPAPVEVEPLRPVLTHAAPAEDTRFRRIMRGGFTDMLRGKAKGAGDTPPSQ

SEQ ID NO: 6 - protein, 1195 aa, Solanum tuberosum
MDVEKSSLCNCVVNFLLEENYLLSAFELLHELLDDGRDDQAIRLKQFFSDPSQFPPDQISRFNSIRVADPQSLLEEKEALEEKLALCEYEFRLSQEDIVQLKSELQKKSQTIPCPISDSKIDPSENHGTDFQQQKREGSFSDLGPLKDNERKDLNYAVKEYLLLAGYRLTAMTLLEEVTDQDLDVQQNSSACIPDALRHYYYQYLSSTSEAAEEKIAMLRKNELLVKENDKLKHEKQSLLKSKDMADAQVTVLAKSLEALQKEMKDKEILVQSLKQSLESQRHELNECRAEITSLKMHIEGARSARNFVASDFEGVDLPSTDSYKEEIKVLQNEIQRLKLATNSLNSESLENINEETRNTCPENEVEKSSDHNVFDDSAGVSSGDLGAAGSQLSMTQTSDSQLLMTQTSADTITEPERVVEVSHDNCVGDKVENVLKHNGELPAEAKGLILKPDNLLVESNAQKIGLGTIQILSDALPKIVPYVLINHREELLPLIMCAIERHPDSSTRDSLTHTLFNLIKRPDEEQRRIIMDACVTLARNVGEMRTETELLPQCWEQINHMYEERRLLVAQSCGELAEFVRPEIRDSLILSIVQQLIEDSATVVREASSHNLALLLPFFPSRDKYFKVEEMMFQLVCDPSGVVVETTIKELVPALVNWGKELDHLLQVLLSHALGSAQRCQPLSGVEGSIESHLRALGERERWNIDVLLRLLTELFPFVRKKAIDTCPFPLVSDDERLVFSTSVLEQYAGGKMDWPSLEWLHIDCFSALIELASLLPQKEDNLRNRITRFLLAVSDLLGEPYLTHIMLPVFLVAVGDDGDLSYFPATYQSRIRGLKPKTAVAERLATIGVLPLLLAGVLGSPRKHELLTEYLRNLLIQTSGQESQTVKREIFFSVRFLCTFDEHHNMIFNILWEMVVSSEINMKATAANLYKVIVPCIDAKVASTHVLPALVTLGSDQNLNVKYASIDAFGAVAQQYKNDAIVDKIRVQMDAFLEDGSHEATLAVVRALVMAVPHTTEGLRDYLLSKIFLLTATPPPSSDMMRRRERANTFCESIRALDATDLSASSVRDFLLPAIQNLLKDADSLDPAHKEALEIVMRERSGGTFDTISKVMGAHLGIASSVSSFFGEGGLLGKREAGDPTSPPPPVEVEPLRPVLTHAAPAEDTRFRRIMRGGFTDMLRGKAKGTEDTPPSQ

SEQ ID NO: 7 - protein, 1183 aa, Oryza sativa
MEATGAGAGEERWASLCNCVVNFLLEERYHLTALELLQELQEDGRHAHALRLRSFFSDPALFPPDLVARASSAPPGADPQSLLEEKIAAEEKLALTDYDLRLAREDLSRLKLELQKQKESSPDGSNATDALTNEGSSHHDKRDAKISTLGPLKDNERKDLNCAVKEYLLLAGYRLAAMTFIEEVPDQDLDVWINSSACVPDALRRYYYQYLSSTTEAAEEKISILRENETLLKDNESLGAEKDALIKSREVANSQIAALRKSLEAAHKDIKEKEKTVQDLKQSLDVQRKELNDCRAEITSLKMHIEGTRSSKRLSSGDTDGLIPANSMEEIVVLSSEHDNLKGSESITSKLASEVSLAEGKKKDHENMESSLEGSPGPEAEVSCSTAENSGYGTSGEDKSGTNTCFEDLSVNGNLHGSGNSQGDSDSISVYLTDDKVHTEKVESPYKQKSSDKMALETIKIVSDALPKIVPYVLINHREELLPLIICAIEKHPDSDVRDSLTHTLFNLIKRPDGQQRRIIMDACVELAKSVGEMRTETELLPQCWEQINHQYEERRLLVAQSCGELAVYVRPEIRDSLILSIVQQLVEDSATVVREAATHNLTLLLPLFPNLDKYYKVEELMFQLVCDPSGAVVNVALKELVPAVVRWGDKLDQILRILLAHILASAQRCPPVSGVEGAIDSHLRVLGEQERWNIDVLLRMLTELLPFIHQKAISTCPFAADPSTGTMPESYFSKSCLKLYAAGDTEWSAFEWMHTECLPDLIKLACLLPAKEDNLRTVIRKYLLDVSGRYGIDYLEHVMLPVFLVAAGDIDSSDFTYFPLATQSRVRGLRPKTSIAEKLGIVCVLPLLLSGILGYPSKRQQLEEYLRKLLIQNTKDGSFSMHHTAEIIDAVRFLCMFEEHHVAVFNIVWEMVVISDANLKTNAAALLKALVPYISVKVASTHVLPALITLGSDQNLTVKYASIEAFGAVAQHFKNDMVVDKIRIQMDAFLEDGSHEATVSVIRALAVAVPHTTDRLREYLLTKIFKLTSAPPTGNDIERRREIANVFCEALRAVDATDLPATSVRDLLLPSIQNLLKDLDALDPAHKEALEVIARERSGGKLESLGKVMGAHLGIASSMSSFFGESSLLVKKESGEQHDTAATTPSQPTPQTQQENTRFGRIMLGGFGDMLRGKAKGSDEPS

SEQ ID NO: 8 - protein, 1179 aa, Sorghum bicolor
MAAVGVGAADERWASLCNCVVNFLLEERYHLTALELMQELQEDGRSAHALRLRAFFSDPALFPPDLVARASSAPPGADPQILLQEKIAAEEKLALVEYDLRLAKEDLSQFKLDLQKQKESSPDDSNGLLLGASIREGSTSQQDKWDTKISALGPLKDNERKDLNCAVKEYLLLAGYRFAAMTFIEEVPDQDLDVWPNSSACVPDALRRYYYQYLSSSAEAAEEKISILQENETLLKDNERLNAENDSLMKSREGANSQVTALRKSLEAAHRDIKDKEKMIQDLRQSLDVQRKELNDCRAEITALKMYIEGTQSSKQLFVGTSDGVKLHSIANSVGASSLNNEDEDSKGSEAVTKKLASAVNITDDTQKDRQVLESSVEGSSISETPVSFTTDENGSYDTSEKDKSASNISSNNVCFQSNLHGASMTGKSQGSSDGISMYLSIEKLESPSKQKCSDKMALETIKIVSDALPKIVPYVLINHREELLPLIICAIEKHPDSDVRDSLTHTLFNLIKRPDGEQRHIIMDACVELAKSIGEMRTETELLPQCWEQINHQYEERRLLVAQSCGELAIYVRPEIRDSLILSIVQQLVEDAAVIVREAATHNLALLLPMFPNLDKYYKVEELMFQLVCDPSGAVVEVALKELVPAVVTWGDKLDQISRVLLAHILASAQRCPPISGVEGTIDSHLRVLGEQERWNIGVLLRMLTELLPFIHQKAIQTCPFASVNPSSTPENFSASCLKSYAAGDSEWSAFEWMHTDCLPNLIKLACLLPVKEDNLRTIITKYLLEVSGLYGKDYLEHIMLPVFLVAAGDIDSGDFTYFPLSIQPKVRGLRPKTSTAEKLAIMCVFPLLLSGILGSPSSRQQLEEYLRKVLIQNTKDGSFSMHHTTEIINAVRFLCLFVEHHSVIFNVLWEMVVSSGTCLKINAAALLRALVPYTDVKVASTHILPALVTLGSDQNLKVKYASIDAFGAVAQHFKNDMVVDKIRIQMDAFLEDGSHEASISVIRALAVAVPHSTDRLREYLLTKIFNMTSITPVSDDIERRCERANVLCEALRALDATDLPAMGVRDLLLPSIQNLLKDLDALDPAHKEALEIISRERSGGTLESISKVMGAHLGIASSVSSFFGESSLLTKKEGGEEHDPAGPTVPEPNLQAQPESTRFGRIMRSGFGDILRGQSKG

SEQ ID NO: 9 - protein, 757 aa, Zea mays
MTGKSQGSSDGISMYLSIEKLESPSKQKSSDKIALETIKIVSDALPKIVPYVLINHREELLPLIICAIEKHPDSDVRDSLTHTLFNLIKRPDGEQRRIIMDACVELAKSVGEMRTETELLPQCWEQINHQYEERRLLVAQSCGELAVYVRPEIRDSLILSIVQQLVEDAATVVREAATHNLALLLPMFPNLDKYYKVEELMFQLVCDPSGAVVEVALKELVPAVVRWGDKLDQISRVLLAHILASAQRCPPISGVEGTIDSHLRVLGEQERWNIGVLLRMLTELLPFIHQKAIQTCPFASADPTSSTPENFSASCLKSYATGDSEWSAFEWMHTDCLPDLIKLACLLPVKEDNLRTIITKYLLEVSGLYGKDYLEHIMLPVFLVAAGDIDSGDFTYFPLSIQPKVRGLRPKTSTAEKLAIMCVFPLLLSGILGSPSSRQQLEEYLRKVLIQNTKDGSFSMHHTTEIINAVRFLCLFVEHHSVIFNILWEMVASSDTSLKINAAALLRALVPYTDVKIASTHILPALVTLGSDQNLKVKYASIDAFGAVAQHFKNDMVVDKIRIQMDAFLEDGSHEASVSVIRALAVAVPHSTDRLREYLLTKIFKMTCITPVGNDIERRRERANVLCEALRALDATDLPAMGIRDLLLPSIQNLLKDLDALDPAHKEALEIISRERSGGTLESISKVMGAHLGIASSVSSFFGENSLLTKKEGGEEHDPAGPTAPEQNLQAQPESTRFGRIMRSGFGDILRGQSKGQ

SEQ ID NO: 10 - protein, 1187 aa, Coffea arabica
MDVEKSSLCNCVVNFLLEENYLLTAFELLHELLDDGRDDHAIRLKEFFSDPSRFPPDQISRLNSLRVADPQSLLDEKEALEEKLALSQYELRLAQEDVLKLKTELENRSKGTLGERSETNLDASADVQRQRNDASFLDLGSLKDNERQDLNCAVKEYLLFAGYRLTAMTFYEEVTDQNLDVWQNSSACVPDALRHYYYQYLSSSTEAAQEKITMLRENESLQKENDKLKKEKQSLLKSKDIADTQVVALMKSLEAFQKDIKDKEVLVQEMKQSMESQRKELNDCRAEITTLKMHIARSGQNLVSSDSKHVELRSSESYMEEIKLLENEIARLKGTNAMNIEPTEAFEQSKGGDAESEVRDSVKANRLESPGHISREDLRSEDSNSQSVPTVDGTTNILDKVSEGKHLSLSDDNGVFVNNEKFLKGVHETPIETNELILRTENIPVDAETTGLATIQVLSDALPKIVPYVLINHREELLPLMMCAIERHPDSMTRDSLTHTLFNLIKRPDEQQRRIIMDACVTLAKNVGEMRTETELLPQCWEQINHMYEERRLLVAQSCGELAEFVRPEIRDSLILSIVQQLIEDPAIVVREAASHNLALLLPLFPNMDKYFKVEEMMFQLVCDPSGVVVETTIKELVPALVKWGNKLDHVLQTLLSHILGSVQHCPPLSGVEGSVESHLRVLGERERWNIDVLLRLLTELLPFLHQKAVQTCPFASVSNGVGTFFSVSLLELYSRGNVEWPSFDWLHIDCFPDLIQLASLLPQKEDNLRNRITKFLLRVSECYGEAYLTHIVLPVFLLAVGDDGDLTYFPQNTHEKIIGLRPKTTVSGRLATMGVLPLLLAGVLGSWSKHDYLTEYLRKKLFQSSEEEIRPAKPELVNSVRFLCMYKDNHNMIFDILWEMVVNSNIDTKICAANLLKSIVPYLDAKIASTHVLPALVTLGSDQNLNVKYASIDAFGAVAQHYKNDMIIDKIRVQMDAFLEDGSHEATIAVVRALVVAVPHTTDRLRDYILYHSALSSLFLCFKCLSSFAVILTVPLPSNDLIRRRERANAFCEAIRALDATDLPASSVRDFLLPGIQNLLKDTDALDPAHKEALEIIMKERSGGTLDTISKVMGAHLGLPSSVSSFFGESGLLGKRETVDQALPSPEPVPSPPAVEDTRLRRIMRSSFTDMLRGKAKGSDDTPHSQ

SEQ ID NO: 11 - protein, 935 aa, Triticum aestivum
MKTSELANNQVAALRKSLDAAHMDIKEKEKMVRDLKQSLDVHRKELNDCRAEITALKMHIEGTQSSKQMPVGDTDVFSTQLNANSMGEAAALINEHENLKGTESITIKLVSAAALTEDTRKDHKNTESTIEGSPDSEAPVPCSTAGGGGYGTSGYESGTDTSPEGISVNGTQHGAGNSQGNSGSISVYVSEDKVNTEIVESPSIHKSSYKMALETIKIVSDALPKIVPYVLINHREELLPLIICAIERHPDSDVRDSLTHTLFNLIKRPDGQQRRIIMDACVELATSVGEMRTETELLPQCWEQINHQYEERRLLVAQSCGELAIYVRPEIRDSLILSIVQQLVEDSATVVREAATHNLALLLPLFPNMDKYYKVEELLFQLVRDPSGVVVDVALRELVPAVVGWGGKLDQILRVLLSHILASAQRCPSVSGVEGTIDSHLRVLGEQERWTIEVLLRMLTELLPFIHQKAIETCPSVDPSENYISESSLKLYATGETEWSAFEWMHTECLPDLIKLACLLPAKEDSLRTVITKYLLAVSGRYGKDYLEHIMLPVFLIAAGDIDSGDFIYFPLSIQSKVRGFRPKTSVAEKIAIICVLPLLLSGILGSPSSRQQLEEYLRKLLIQNTKDGSFSMHHTAEIIDAIRFLCIFEEHHGVIFHILWEMVVSSDTNLKTSAAALLKALVPYVGVKVASTHVLPALITLGSDQNSAVKYASIDALGAVAQHFKSDMVVDKIHIQMDAFLEDGSHEATISVIRALAVAVPHSTDRLREYLLTKIFRLTSAPPTENNVERRRERANVFCEALRALDATDLPASSVRDLLLPSIQNLLKDVDALDPAHKEALEVIGRERSGGTLESIGKAMGAHLGIATSVSSFFGESSLLGKKEGGEQHDPAAASVPPQVGLQTQQENTRFGRIMRGGFGDMLRGKAKGGDEPS

SEQ ID NO: 12 - protein, 1188 aa, Citrus X sinensis
MDVERSSLCNCVVNFLLEEKYLLTAFELLQELLDDGRDAQAIRLKEFFSDPSHFPPDLITRFNSLRVADPQSLLEEKEALVEKLAITEYELRLAQEDVTKLKAELKKKSDSSLENLNESNENNHGNHGVEFQRQKRDISFSDLGPLKDIERKDLNCAVKEYLLLAGYRLTAMTFYEEVTDQNLDIWQNTPARVPDALRHYYYQYLSSTTEAAEEKIAMLRENESLLKVNERLNHEKESLLKTKEISDGQISALTKSLEALHRDLKDKENLILDLKKTTEHQRRELNDCTAEITALKMHIEGSHSVRNFATTNGDVIQSQPVERYEEEIKSLLKEIERLRAKSTNASDSLGSVYSESMQTEEKVVEVDEDKTVLAHPSVEVVNSEDAQSLATQTPDNNTAKQPNEVLQGESTSSLKENIASENSENVPNLNDESPLKDSGLPLQSDNASLEAASDKMGLGTIQILADALPKIVPYVLINHREELLPLIMCAIERHPDTSTRDSLTHTLFNLIKRPDEKQRRIIMDACVTLAKNVGEMRTEMELLPQCWEQINHMYEERRLLVAQSCGELAEFVRPEIRDSLILSIVQQLVEDSATVVREAAARNLALLLPLFPNTDKYFKVEDLMFQLVCDPSGVVVETTFKELLPAVINWGSKLDHILRVLLSYILSSAQRCPPLSGVEGSVESHLRVLGERERWNLEVLLRMMAELLPFMQKNAIETCPFSSVSLSEETVFPSSLLELYAGGHIEWPAFEWMHVDCFPGLIQLACLLPEKEDNLRNRITKFLLAVSKQFGDSYLTHIMLPVFMVAVGDNANLTFFPSTIHSGIRGLKPRTAVGERLATMGVLPLLLAGVLGAPSKHDQLADYLRKLLVEGTMKENHTVKCNAEIVNAVRFLCTFEEHHTMVFNILWEMVVSSNIDMKINAANLLKVIVPYINAKVTSMQVLPALVTLGSDQNLNVKYASIDAFGAVAQHFKNDMIVDKIRVQMDAFLEDGSHEATVAVVRALAVAVPHTTERLRDYLLSKIFQLSAVPSSSSDVMRRRERANAFCESIRALDATELSATSVRDFLLPAIQNLLKDADSLDPAHKEALEIIMKDRSGGTLETISKVMGAHLGITSSVTSFFGGGVGEGLLGKKEIAEQSAEPVHSPEPPPPAPAEDTRFMRIMRGNFVGDMLRGKAKTSEDTSRTQNQ

SEQ ID NO: 13 - protein, 1156 aa, Malus domestica
MDVERSSLCNCVVNFLLEENYLLTAFELLHELLDDGRDDQAIRLKDFFADSTQFPPDQISRFNSLRVADPQCLLEEKEAVEEKLAISEYELRLAQEDISKLKAELQKKAESPSNELRDSNANVSVNNGPEFQRQKRDVSFSDLGLLKDNERRDLNCAVKEYLLIAGYRLTAMTFFEEVTDQNLDVWKNSPACVPDALRHYYYQYLSSTTEAAEEKISMLRENDSLSKEKESLYLEKLSLLKNKDLAEGQISTLIKSLEALQKDLKDKENLVQDLKKSLEHQRKELNDCRAEVTALKMHIEGYRSGRNMVAADADQIQPLSLEKYKEEIKSLQMELESLKSKNAKANDYSDSTNFGKEPVQMEEKVVVVDEDKSIIPAIDVESRVVEKEEDQSLAAQTFHDNTVKPKEVSHEVSVGVLSDSSTLVNGDSVSRQNGEPSSGSSLHLTSEKLSPEDVSEKRGLETIQILADALPKIVPYVLINHREELLPLIMCAIERHPDSSTRDSLTHTLFNLIKRPDEQQRRIIMDACVTLAKDVGEMRTETELLPQCWEQINHMYEERRLLVAQSCGELAEFVRPEIRDSLILSIVQQLIEDSATIVREAAAHNLALLLPLFPNMDKYYKVEDLMFQLVCDPSGVVVETTLKQLVPAVNKWGNKLDHILRVLLSHISSSVQRCPPLSGVEGSVESHLRVLGERERWNVDVLLRMLVEIIPYVHQKAIEMCPVSSDPETTGTIFSKSFLELYAGGHVQLPAFEWLHVDCFPALIQLACLLPPKEDNLRNQITKFLLAVSELYGDSYLTHIMLPVFLVAVGEDAELTFFPSATHSGIKGLRPRTAVANRLATMCVLPLLLAGVLGGPSKHEQLLEYLRKLLVEGVANQSTKCNAEIVDAVRFLCTFEDHHGMIFNLLWEMVVSSNIDMKINAANLLKVIVPYIDAKVASTHILPALVTLGSDQNLSVKYASIDAFGAVAQHFKNDMIVDKIRVQMDAFLEDGSHEATIAVIFQLTATPPSSDLMRRRERANAFCEAIRALDATDISATSVRDFLMPAIQNLLRDTDALDPAHKEALEIVMKERSGGTFDTISKVMGAGLASSVSSFFGEGGLLGKKESPEPPPEPVESPKAAPLPPAEDTRLRRIMRGNFTDMLRGKARGPEETQNQ

(19) United States
(12) Patent Application Publication
(10) Pub. No.: US 2026/0242816 A1
(43) Pub. Date: Aug. 20, 2026

(54) COMPOSITIONS AND METHODS FOR IMPROVED DELIVERY OF ENDOGENOUS ANTIMICROBIAL PROTEINS IN PLANTS

(71) Applicant: BOARD OF TRUSTEES OF MICHIGAN STATE UNIVERSITY, East Lansing, MI (US)

(72) Inventors: Federica Brandizzi, Okemos, MI (US); Deepak D. Bhandari, East Lansing, MI (US)

(21) Appl. No.: 19/355,799

(22) Filed: Oct. 10, 2025

Related U.S. Application Data
(60) Provisional application No. 63/705,900, filed on Oct. 10, 2024.

(51) Int. Cl. C12N 15/82; C07K 14/415

(52) U.S. Cl. CPC C12N 15/8281; C07K 14/415; C12N 15/8282

(57) ABSTRACT

Disclosed herein are plants, plant seeds, and plant cells that are modified to overexpress a trans-Golgi network (TGN) associated and microtubule (MT)-binding protein, TGNap-1. Such plants, plant seeds, and plant cells can be, but are not limited to, Arabidopsis thaliana, grape, cotton, soybean, tomato, potato, rice, sorghum, corn, coffee, wheat, citrus (e.g. orange), an apple. The modified plants exhibit approximately a five-fold reduction against the plant pathogen Pseudomonas syringae DC3000, a rod-shaped, gram-negative bacterium. The modified plants also exhibit normal plant size and biomass accumulation as compared to a control plant.

### CROSS REFERENCE TO RELATED APPLICATIONS

This application claims the benefit of U.S. Provisional Patent Application Ser. No. 63/705,900, filed on Oct. 10, 2024, the disclosure of which is incorporated herein by reference in its entirety.

### FEDERAL FUNDING

This invention was made with government support under Grant Nos. DE-FG02-91ER20021, DE-AC02-76SF00515, and DE-SC0018409 awarded by the Department of Energy. The Government has certain rights in the invention.

### INCORPORATION OF SEQUENCE LISTING

The instant application contains a Sequence Listing named “P14958US01” which has been submitted electronically in XML format, is 26,955 bytes in size, was created on Oct. 10, 2025, and is herein incorporated by reference in its entirety.

### TECHNICAL FIELD

Disclosed herein are novel plants, plant parts, and amino acid sequences for overexpressing TGNap1, along with methods of making the same by growing a plant or lot, and methods of using the same.

### BACKGROUND

Biotic diseases lead to major crop losses worldwide and thus reduce food production. Therefore, understanding the fundamental mechanisms underpinning plant defense is critical to designing sustainable crop management systems. Over the course of evolution, plant-pathogen interactions have shaped the plant immune system against diverse pathogens and led to the development of bacterial strategies to suppress plant immunity.

Current strategies for engineering crop resistance are limited because plant immunity and growth are often inversely linked. The development of pathogen-resistant crops that maintain optimal growth is needed.

### SUMMARY

Disclosed herein are plants, plant seeds, and plant cells that are modified to overexpress a trans-Golgi network (TGN) associated and microtubule (MT)-binding protein, TGNap-1. Such plants, plant seeds, and plant cells can be, but are not limited to, Arabidopsis thaliana, grape, cotton, soybean, tomato, potato, rice, sorghum, corn, coffee, wheat, citrus (e.g. orange), and/or an apple seed or plant. The modified plants exhibit approximately a five-fold reduction against the plant pathogen Pseudomonas syringae DC3000, a rod-shaped, gram-negative bacterium. The modified plants also exhibit normal plant size and biomass accumulation. Methods of making and using such plants, plant seeds, and plant cells are also described herein.

### BRIEF DESCRIPTION OF THE DRAWINGS

FIG. 1 is a sequence alignment of TGNap1 homologs.

FIGS. 2A-2D: Four-week-old Arabidopsis plants of the indicated genotypes were infiltrated with Pst DC3000 (FIG. 2A), Pst AvrRps4 and Pst AvrRpt2 (FIG. 2B), Pst hrcC (FIG. 2C). Bacterial titers were determined at 3 dpi. Data from at least three independent experiments are represented in a boxplot with data points (technical replicates). Individual data points are represented with different shapes. Statistical differenTGNap1 between genotypes were analyzed using ANOVA (Tukey's HSD, P<0.05). FIG. 2D is a Western blot showing accumulation of TGNap1-YFP in four-week-old leaves of TGNap1-YFP transgenic plants treated with mock (10 mM MgCl₂) and Pst DC3000 (6 h and 24 h) probed with α-GFP serum. Tgnap1-2 leaves were used as control to ascertain the specificity of protein bands. Ponceau red was used as loading control. Experiments were repeated twice with similar results.

FIGS. 3A-3C: FIG. 3A is a graph showing PR/expression in leaves treated with either mock or Pst DC3000 (Pst) and analyzed with qRT-PCR. Data from three independent experiments are represented in a boxplot. *** indicates significant differenTGNap1 (Student's/test) with the respective mock-treated genotypes. FIG. 3B shows immunoblot analyses of leaf apoplastic fluids that were isolated 24 hpi with either mock or Pst DC3000. Total and apoplastic protein samples were probed with α-PR1, α-PR2, and α-cFBPase. A quantification of PR1, PR2 protein levels in Pst DC3000-treated apoplastic samples, normalized to Col-3 is represented as a barplot. Experiments were repeated three times with similar results. FIG. 3C shows leaf apoplastic fluids that were isolated 24 hpi with either mock or Pst DC3000. Isolated apoplastic fluid were subjected to label-free LC-MS analysis. Proteins detected with at least two peptides with a 1% FDR were selected for analysis. Proteins were filtered with a cutoff of P<0.05 (Benjamini-Hochberg corrected). Selected proteins have been highlighted. L2FC Log2 fold change.

FIG. 4 shows a barplot of Pst DC3000 bacterial growth. Arabidopsis plants of the indicated genotypes: wild type (Col-3), complete TGNap1 knockout (tgnap1-2), and TGNap1-YFP transgenic plants were infiltrated with Pst DC3000 and bacterial growth was measured.

FIG. 5 is a vector map depicting Arabidopsis TGNap1 fused to m Venus (TGNap1-Venus) driven by the 35s promoter.

FIG. 6 shows RT-PCR analysis (25, 30, and 35 PCR cycles) of TGNap1-Venus, Actin-41 was used as a house-keeping gene.

FIG. 7: shows fresh fruit weight (grams) of three Tomato transgenic lines expressing Arabidopsis TGNap1 (tomTGN #4, tomTGN #5, tomTGN #6) compared to Wild Type (WT). Differences between lines were measured using Anova (Tukey HSD α<0.05)

FIGS. 8A and 8B show quantification of late blight resistance of a tomato transgenic line expressing Arabidopsis TGNap1. FIG. 8A shows pictures of macroscopic disease phenotypes of wildtype (WT) and TGN #5 leaves infected with Phytophthora infectants at 5 dpi. FIG. 8B is a graph showing quantification of lesion diameter of leaves infected in FIG. 8A. Difference between genotypes were measured using Anova (Tukey HSD α<0.05). TomTGN lines exhibit increased resistance to Phytophthora infestans (late blight).

FIG. 9 shows pictures of plant growth of Wild Type (WT) tomato and TGNap1 mutant tomato lines (TomTGN).

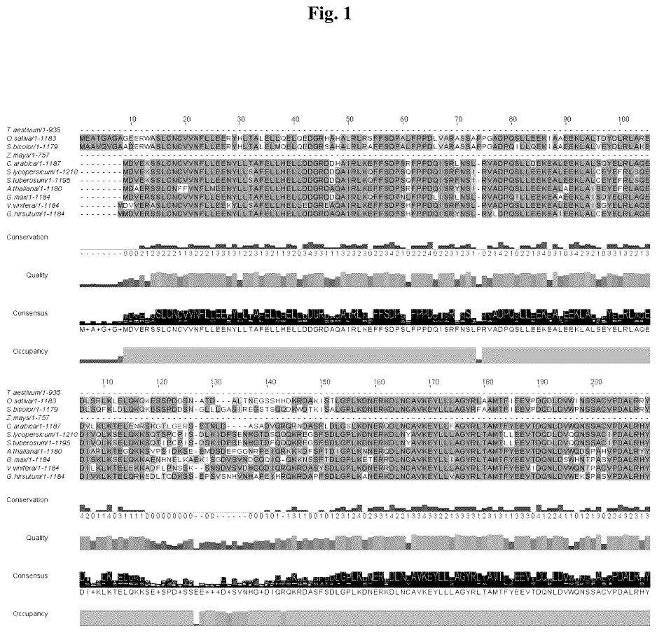

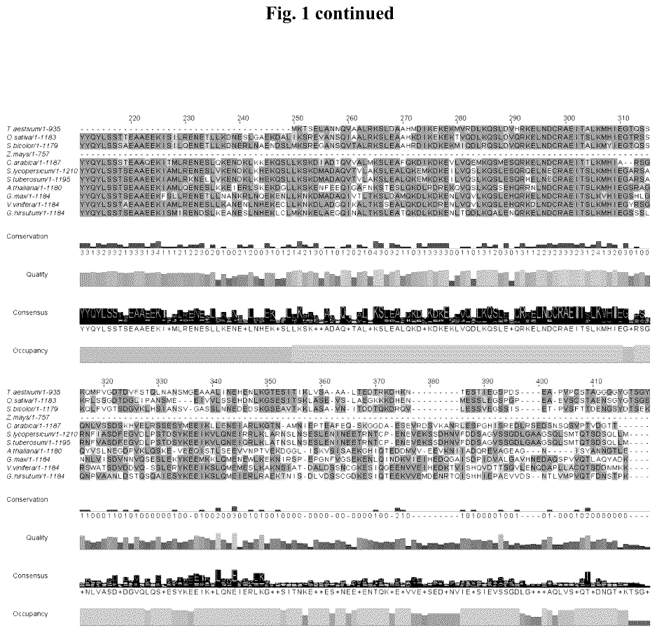

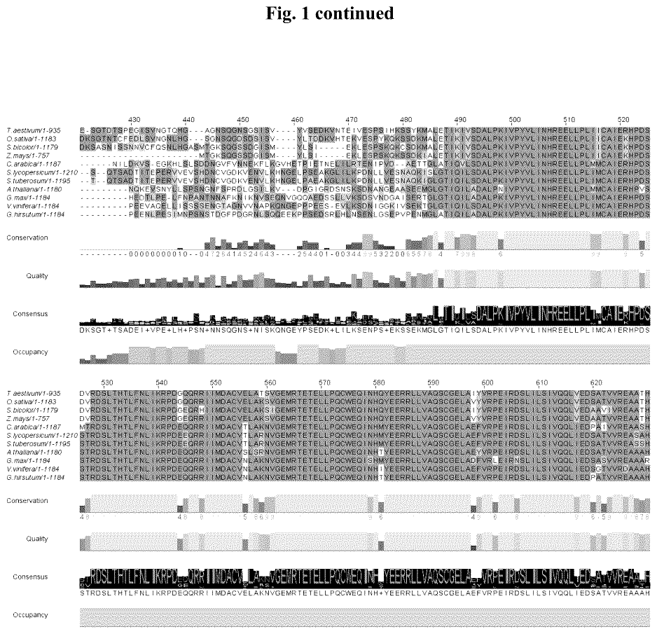

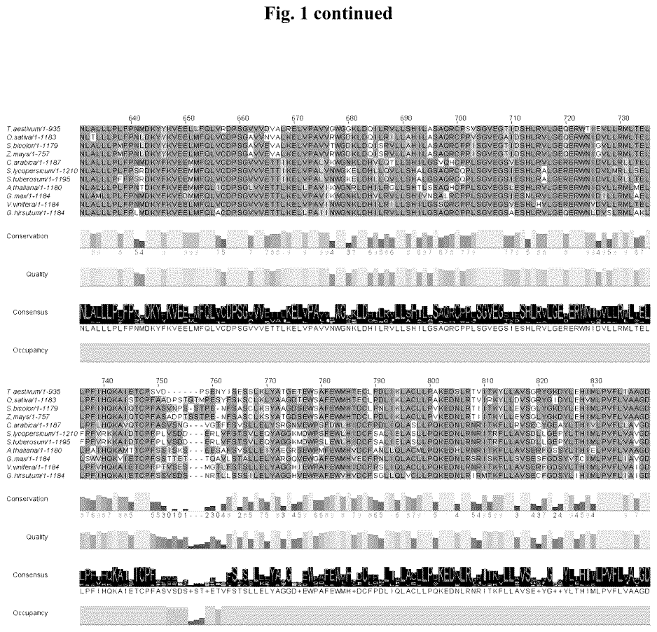

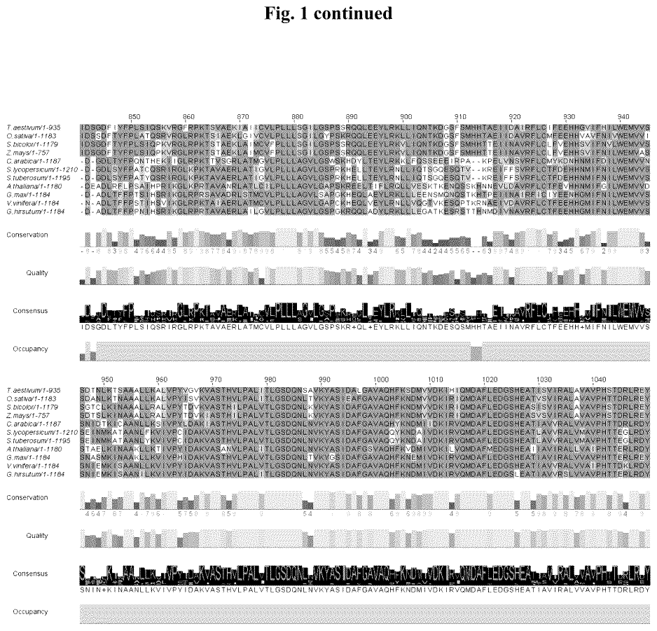

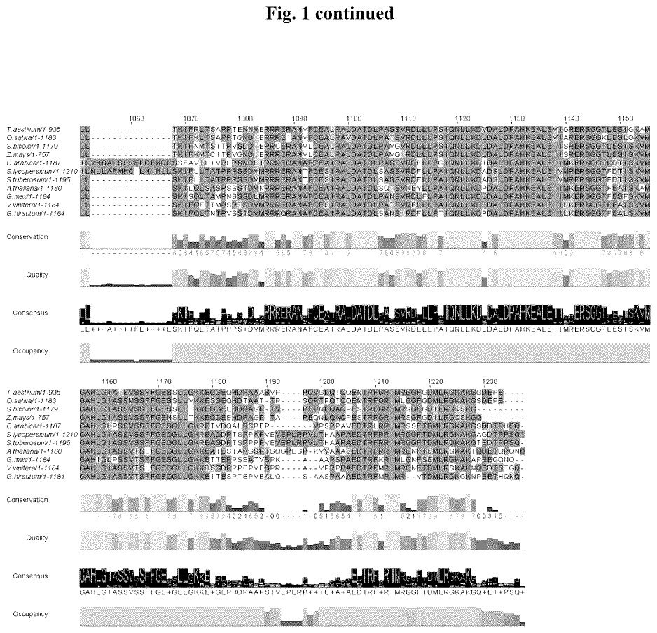

### DETAILED DESCRIPTION

Unless defined otherwise, all technical and scientific terms used herein have the same meaning as commonly understood by one of skill in the art to which the disclosure pertains. Although any methods and materials similar to or equivalent to those described can be used in the practice or testing of the described refrigerating methods, it is the preferred methods and materials that are described.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “control” refers to a reference standard where one or more treatments are omitted.

As used herein, the phrase “gene-editing” includes genome modification by homology directed repair (HDR) or by non-homologous end-joining (NHEJ) mechanisms. Such gene-editing includes embodiments where a site specific (sequence specific) nuclease and a donor template are provided.

As used herein, an “exogenous” agent or molecule refers to any agent or molecule from an external source that is provided to or introduced into a system, composition, plant cell culture, reaction system, or plant cell. In certain embodiments, the exogenous agent (e.g., polynucleotide, protein, or compound) from the external source can be an agent that is also found in a plant cell. In certain embodiments, the exogenous agent (e.g., polynucleotide, protein, or compound) from the external source can be an agent that is heterologous to the plant cell.

As used herein, a “heterologous” agent or molecule refers: (i) to any agent or molecule that is not found in a wild-type, untreated, or naturally occurring composition or plant cell; and/or (ii) to a polynucleotide or peptide sequence located in, e.g., a genome or a vector, in a context other than that in which the sequence occurs in nature. For example, a promoter that is operably linked to a gene other than the gene that the promoter is operably linked to in nature is a heterologous promoter.

As used herein, the terms “include,” “includes,” and “including” are to be construed as at least having the features to which they refer while not excluding any additional unspecified features.

As used herein, there terms “ortholog” or “orthologous” refer to genes and/or encoded proteins from different species that have the similar or identical functions (e.g., exhibit similar or identical phenotypes when suppressed and/or overexpressed). Orthologous genes and their encoded proteins will typically exhibit a certain degree of sequence conservation and a similar pattern of expression (e.g., tissue, temporal, and/or cell cycle stage specific expression). Sequence conservation in orthologous genes and their encoded proteins can extend over the entire polynucleotide or amino acid sequence or can be limited to certain functional domains (e.g., transcription activation, DNA, protein, substrate, and or membrane binding, dimerization, oligomerization) of the encoded protein or residues located therein and the corresponding polynucleotide coding sequence. In certain embodiments, sequence conservation in an orthologous gene to the gene can be at least 40%, 50%, 60%, 70%, 80%, 85%, 90%, 95%, 98%, or 99% over the entire polynucleotide or amino acid sequence or to portions of a polynucleotide or amino acid sequence which respectively encode or comprise certain functional domains (e.g., pRb binding motif or domain including an LXCXE motif and/or a helix 4 motif).

As used herein, the term “overexpressed” where used herein with regards to genes refers to production of the protein encoded by the gene in a modified plant in an amount that is increased in comparison to the amount found in an unmodified plant cell or plant.

The term “polynucleotide” where used herein is a nucleic acid molecule containing 2 or more nucleotide residues. Polynucleotides are generally described as single- or double-stranded. Where a polynucleotide contains double-stranded regions formed by intra- or intermolecular hybridization, the length of each double-stranded region is conveniently described in terms of the number of base pairs. Embodiments of the systems, methods, and compositions provided herein can employ or include: (i) one or more polynucleotides of 2 to 25 residues in length, one or more polynucleotides of more than 26 residues in length, or a mixture of both. Polynucleotides can comprise single- or double-stranded RNA, single- or double-stranded DNA, double-stranded DNA/RNA hybrids, chemically modified analogues thereof, or a mixture thereof. In certain embodiments, a polynucleotide can include a combination of ribonucleotides and deoxyribonucleotides (e.g., synthetic polynucleotides consisting mainly of ribonucleotides but with one or more terminal deoxyribonucleotides or synthetic polynucleotides consisting mainly of deoxyribonucleotides but with one or more terminal dideoxyribonucleotides), or can includes non-canonical nucleotides such as inosine, thiouridine, or pseudouridine. In certain embodiments, the polynucleotide includes chemically modified nucleotides (see, e.g., Verma and Eckstein (1998) Annu. Rev. Biochem., 67:99-134). Chemically modified nucleotides that can be used in the polynucleotides provided herein include: (i) phosphorothioate, phosphorodithioate, or methylphosphonate internucleotide linkage modifications of the phosphodiester backbone; (ii) nucleosides comprising modified bases and/or modified sugars; and/or (iii) detectable labels including a fluorescent moiety (e.g., fluorescein or rhodamine or a fluorescence resonance energy transfer or FRET pair of chromophore labels) or other label (e.g., biotin or an isotope). Polynucleotides provided or used herein also include modified nucleic acids, particularly modified RNAs, which are disclosed in U.S. Pat. No. 9,464,124, which is incorporated herein by reference in its entirety.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Pathogen-induced crop losses pose a significant challenge to global agriculture, with an estimated 40% of global crop production lost annually to pests and pathogens. Current strategies for engineering crop resistance are limited because plant immunity and growth are often inversely linked. However, we have identified a gene in Arabidopsis, TGNap1, which controls the transport of a specific subset of antimicrobial proteins. Overexpression of TGNap1 in Arabidopsis resulted in an approximately five-fold reduction in pathogen growth as compared to control plants, without compromising plant size and biomass accumulation. The invention bypasses the traditional bottleneck of defense-growth by genetically engineering TGNap1 into wild-type Arabidopsis. This modification enhanTGNap1 plant resilience to pathogens while maintaining optimal growth independently of the salicylic-acid regulated defense pathway, providing a novel, complementary strategy for pathogen resistance. This invention offers a straightforward strategy for breeding pathogen-resistant crops, addressing a bottleneck in agricultural biotechnology and opening additional avenues for enhancing crop resilience.

As described herein, the inventors show that overexpression of TGNap1 can reduce growth of bacterial plant pathogens on the transgenic plant by approximately five-fold as compared to the control plant. Examples of TGNap1 protein homologs are provided in SEQ ID NO: 1-13. In various embodiments, TGNap1 can be overexpressed by 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, or more as compared to a control plant.

Plants, plant seeds, and plant cells overexpressing TGNap1 may be resistant to a variety of plant pathogens. In various embodiments, the bacterial plant pathogens or other plant pathogens comprise Pseudomonas syringae (e.g., DC3000 or other strains), powdery mildew, and/or Oomycete pathogen (downy mildew).

Plants, plant seeds, and plant cells described herein comprise Arabidopsis thaliana, grape, cotton, soybean, tomato, potato, rice, sorghum, corn, coffee, wheat, citrus (e.g. orange), or an apple seeds or plants. Also provided herein are biological samples obtained from any of the transgenic plant, plant seed, or plant cells described herein, wherein the biological sample comprises an endogenous or heterologous promoter operably linked to the nucleic acid segment encoding the endogenous TGNap1 protein. The biological sample can comprise grain meal or a plant tissue homogenate.

### Transformation of Plant Cells

Plant cells can be modified to include expression cassettes or transgenes that can express any of the TGNap1 proteins described herein. Such an expression cassette or transgene can include a promoter operably linked to a nucleic acid segment that encodes any of the TGNap1 proteins described herein.

Promoters provide for expression of mRNA from the TGNap1 nucleic acids. A TGNap1 nucleic acid is operably linked to the promoter, for example, when it is located downstream from the promoter.

In some cases, the promoter can be a TGNap1 native promoter. However, the promoter can in some cases be heterologous to the TGNap1 nucleic acid segment. In other words, such a heterologous promoter may not be naturally linked to such a TGNap1 nucleic acid segment. Instead, some expression cassettes and expression vectors have been recombinantly engineered to include a TGNap1 nucleic acid segment operably linked to a heterologous promoter. Hence, the promoter can be heterologous to the nucleic acid segment that encodes the TGNap1 protein. The nucleic acid segment that encodes the TGNap1 protein can also be heterologous to the promoter.

A variety of promoters can be included in the expression cassettes and/or expression vectors. In some cases, the endogenous TGNap1 promoter can be employed. Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences can also contain regulatory sequences such as enhancer sequences that can influence the level of gene expression. Some isolated promoter sequences can provide for gene expression of heterologous DNAs, that is a DNA different from the native or homologous DNA. In certain embodiments, overexpression of TGNap1 can be achieved an insertion of one or more transcriptional enhancers in the endogenous TGNap1 gene promoter located at its native location in the plant genome by gene editing. In certain embodiments, the insertion is effected with a DNA donor template comprising one or more enhancers which can be integrated into a specific site in the endogenous TGNap1 gene promoter by gene editing.

Promoters can be strong or weak, or inducible. A strong promoter provides for a high level of gene expression, whereas a weak promoter provides for a very low level of gene expression. An inducible promoter is a promoter that can turn on and off gene expression in response to an exogenously added agent, or in response to an environmental stimulus, or in response to a developmental stimulus. For example, a bacterial promoter such as the Ptac promoter can be induced to vary levels of gene expression depending on the level of isothiopropylgalactoside added to the transformed cells. Promoters can also provide for tissue specific or developmental regulation. Hence, in some cases, the promoter within such expression cassettes/vectors can be functional during plant development or growth. A strong promoter for heterologous DNAs can also be used and, in some cases, such a strong promoter can be advantageous because it provides for a sufficient level of gene expression for easy detection and selection of transformed cells and provides for a high level of gene expression when desired.

Expression cassettes/vectors can include, but are not limited to, a plant promoter such as the CaMV 35S promoter (Odell et al., Nature. 313:810-812 (1985)), or others such as CaMV 19S (Lawton et al., Plant Molecular Biology. 9:315-324 (1987)), nos (Ebert et al, Proc. Natl. Acad. Sci. USA. 84:5745-5749 (1987)), Adh1 (Walker et al., Proc. Natl. Acad. Sci. USA. 84:6624-6628 (1987)), sucrose synthase (Yang et al., Proc. Natl. Acad. Sci. USA. 87:4144-4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)) or those associated with the R gene complex (Chandler et al., The Plant Cell. 1:1175-1183 (1989)). Further suitable promoters include the poplar xylem-specific secondary cell wall specific cellulose synthase 8 promoter, cauliflower mosaic virus promoter, the Z10 promoter from a gene encoding a 10 kD zein protein, a Z27 promoter from a gene encoding a 27 kD zein protein, inducible promoters, such as the light inducible promoter derived from the pea rbcS gene (Coruzzi et al., EMBO J. 3:1671 (1971)) and the actin promoter from rice (McElroy et al., The Plant Cell. 2:163-171 (1990)). Seed specific promoters, such as the phaseolin promoter from beans, may also be used (Sengupta-Gopalan, Proc. Natl. Acad. Sci. USA. 83:3320-3324 (1985). Other promoters useful in the practice of the invention are available to those of skill in the art.

Alternatively, novel tissue specific promoter sequences may be employed in the practice of the present invention. cDNA clones from particular tissues can be isolated and those clones that are expressed specifically in that tissue are identified, for example, using Northern blotting, Preferably, the gene isolated is not present in a high copy number but is relatively abundant in specific tissues. The promoter and control elements of corresponding genomic clones can then be localized using techniques available to those of skill in the art.

A TGNap1 nucleic acid can be combined with the promoter by available methods to yield an expression cassette or transgene, for example, as described in Sambrook et al. (MOLECULAR CLONING: A LABORATORY MANUAL. Second Edition (Cold Spring Harbor, NY: Cold Spring Harbor Press (1989); MOLECULAR CLONING: A LABORATORY MANUAL. Third Edition (Cold Spring Harbor, NY: Cold Spring Harbor Press (2000)). Briefly, a plasmid containing a promoter such as the 35S CaMV promoter can be constructed as described in Jefferson (Plant Molecular Biology Reporter 5:387-405 (1987)) or obtained from Clontech Lab in Palo Alto, California (e.g., pBI121 or pBI221). Typically, these plasmids are constructed to have multiple cloning sites having specificity for different restriction enzymes downstream from the promoter. The TGNap1 nucleic acids can be subcloned downstream from the promoter using restriction enzymes and positioned to ensure that the DNA is inserted in proper orientation with respect to the promoter so that the DNA can be expressed as sense or antisense RNA. Once the TGNap1 nucleic acid is operably linked to a promoter, the expression cassette so formed can be subcloned into a plasmid or other vector (e.g., an expression vector).

In some embodiments, a cDNA clone encoding a TGNap1 protein is synthesized, isolated, and/or obtained from a selected cell. In other embodiments, cDNA clones from other species (that encode a TGNap1 protein) are isolated from selected plant tissues. For example, the nucleic acid encoding a TGNap1 protein can be any nucleic acid with a coding region that hybridizes to SEQ ID NO: 1-13 and that has TGNap1 activity. In another example, the TGNap1 nucleic acid can encode a TGNap1 protein with an amino acid sequence that has at least 90%, or at least 95%, or at least 96%, or at least 97%, at least 98%, or at least 99% sequence identity to SEQ ID NO:1-13. Using restriction endonucleases, the entire coding sequence for the TGNap1 nucleic acid is subcloned downstream of the promoter in a 5′ to 3′ sense orientation. TGNap1 homologs comprise conserved LisH domains. LisH domains are found in microtube (MT)-interacting proteins and facilitate direct protein interaction. See: Renna, L., Stefano, G., Slabaugh, E. et al. TGNap1 is required for microtubule-dependent homeostasis of a subpopulation of the plant trans-Golgi network. Nat Commun 9, 5313 (2018). https://doi.org/10.1038/s41467-018-07662-4 (which is incorporated herein by reference in its entirety).

In some cases, the endogenous TGNap1 gene can be deleted and plant cells with such a deleted endogenous TGNap1 gene can be transformed to include a TGNap1 transgene, for example, by transformation of the plant cells with a TGNap1 expression cassette or expression vector.

The frequency of occurrence of cells taking up exogenous (foreign) DNA can sometimes vary. However, certain cells from virtually any dicot or monocot species can be stably transformed, and these cells can be regenerated into transgenic plants, through the application of the techniques disclosed herein and those available to one of skill in the art. The plant cells, plants, and seeds can therefore be monocotyledons or dicotyledons.

The cell(s) that undergo transformation may be in a suspension cell culture or may be in an intact plant part, slidh as an immature embryo, or in a specialized plant tissue, such as callus, such as Type I or Type II callus.

Transformation of the cells of the plant tissue source can be conducted by any method available to those of skill in the art. Examples are: Transformation by direct DNA transfer into plant cells by electroporation (U.S. Pat. Nos. 5,384,253 and 5,472,869, Dekeyser et al., The Plant Cell. 2:591 602 (1990)); direct DNA transfer to plant cells by PEG precipitation (Hayashimoto et al., Plant Physiol. 93:857 863 (1990)); direct DNA transfer to plant cells by microprojectile bombardment (McCabe et al., Bio/Technology. 6:923 926 (1988); Gordon Kamm et al., The Plant Cell. 2:603 618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli derived plasmid cloning vector. In the case of viral vectors, it is desirable that the system retain replication functions, but lack functions for disease induction.

One method for dicot or monocot transformation, for example, involves infection of plant cells with Agrobacterium tumefaciens using the leaf disk protocol (Hoesch et al., Science 227:1229 1231 (1985). For example, fiber-producing plants (e.g., dicots) such as cotton, flax, hemp, jute, sisal, poplar, and eucalyptus can be transformed via use of Agrobacterium tumefaciens. Arabidopsis is a dicot that is useful for experimental purposes.

Monocots such as Zea mays can be transformed via microprojectile bombardment of embryogenic callus tissue or immature embryos, or by electroporation following partial enzymatic degradation of the cell wall with a pectinase containing enzyme (U.S. Pat. Nos. 5,384,253; and 5,472,869). For example, embryogenic cell lines derived from immature Zea mays embryos can be transformed by accelerated particle treatment as described by Gordon Kamm et al. (The Plant Cell. 2:603 618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. application Ser. No. 08/112,245 and PCT publication WO 95/06128. Furthermore, methods for transformation of monocotyledonous plants utilizing Agrobacterium tumefaciens have been described by Hiei et al. (European Patent 0 604 662, 1994) and Saito et al. (European Patent 0 672 752, 1995).

Methods such as microprojectile bombardment or electroporation are carried out with “naked” DNA where the expression cassette may be simply carried on any convenient plasmid cloning vector. In some cases, it may convenient to use an E. coli derived plasmid or cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Selection of tissue sources for transformation of monocots is described in detail in U.S. application Ser. No. 08/112,245 and PCT publication WO 95/06128.

The transformation is carried out under conditions directed to the plant tissue of choice. The plant cells or tissue are exposed to the DNA or RNA carrying the targeting vector and/or other nucleic acids for an effective period of time. This may range from a less than one second pulse of electricity for electroporation to a 2-3 day co-cultivation in the presence of plasmid bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

Where one wishes to introduce DNA by means of electroporation, itis contemplated that the method of Krzyzek et al., (U.S. Pat. No. 5,384,253) may be advantageous. In this method, certain cell wall degrading enzymes, such as pectin degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic cells were bombarded with intact cells of the bacteria E. coli or Agrobacterium tumefaciens containing plasmids with either the β-glucoronidase or bar gene engineered for expression in maize. Bacteria were inactivated by ethanol dehydration prior to bombardment. A low level of transient expression of the β-glucoronidase gene was observed 24-48 hours following DNA delivery. In addition, stable transformants containing the bar gene were recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, is that the isolation of protoplasts (Christou et al., PNAS. 84:3962 3966 (1987)), the formation of partially degraded cells, or the susceptibility to Agrobacterium infection is not required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon Kamm et al., The Plant Cell. 2:603 618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing damage inflicted on the recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. The techniques set forth here can provide up to 1000 or more foci of cells transiently expressing a marker gene. The number of cells in a focus which express the exogenous gene product 48 hours post bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA microprojectile precipitate or those that affect the path and velocity of either the macroprojectiles or microprojectiles. Biological factors include all steps involved in manipulation of cells before and immediately after bombardment, the osmotic adjustment of target cells to help alleviate the trauma associated with bombardment, and the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

Examples of plants and/or plant cells that can be modified as described herein include Arabidopsis thaliana, grape, cotton, soybean, tomato, potato, rice, sorghum, corn, coffee, wheat, citrus (e.g. orange), or an apple seeds or plants. Examples of plants and/or plant cells that can be modified as described herein can also include fiber-producing, alfalfa (e.g., forage legume alfalfa), algae, apple, avocado, balsam, barley, broccoli, Brussels sprouts, cabbage, canola, cassava, cauliflower, cocoa, tole vegetables, collards, corn, cotton, cottonwood, crucifers, earthmoss, eucalyptus, grain legumes, grasses (e.g., forage grasses), hemp, jatropa, kale, kohlrabi, maize, miscanthus, moss, mustards, nut, nut sedge, oats, oil firewood trees, poplar, sorghum, sunflower, switchgrass, tobacco, tomato, turnips, and wheat. In some embodiments, the plant is a Brassicaceae or other Solanaceae species. In some embodiments, the plant or cell can be a fiber-producing plant, plant seed, or plant cell such as a cotton, hemp, poplar, or eucalyptus plant, seed, or cell. In some cases, the plant, seed, or plant cell can be a cotton species. In some cases, the plant, seed, or plant cell can be a tree species. In some embodiments, the plant, plant seed, and plant cell is not a species of Arabidopsis, for example, in some embodiments, the plant, plant seed, or plant cell is not an Arabidopsis thaliana plant, plant seed, or plant cell.

An exemplary embodiment of methods for identifying transformed cells involves exposing the bombarded cultures to a selective agent, such as a metabolic inhibitor, an antibiotic, herbicide or the like. Cells which have been transformed and have stably into grated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including but not limited to filters and solid culture medium.

Bialaphos and glyphosate are provided as examples of agents suitable for selection of transformants, but the technique of this invention is not limited to them.

An example of a screenable marker trait is the red pigment produced under the control of the R-locus in maize. This pigment may be detected by culturing, cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by deviTGNap1 that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations that provide 100% inhibition followed by screening of growing tissue for expression of a screenable marker gene such as luciferase would allow one to recover transformants from cell or tissue types that are not amenable to selection alone.

### Gene Editing

Embodiments include aforementioned systems, methods, and compositions comprising a gene editing molecule such as an RNA guide for an RNA-guided nuclease (or a polynucleotide encoding an RNA guide for an RNA-guided nuclease); an RNA-guided DNA nuclease (or a polynucleotide encoding an RNA-guided DNA nuclease); an RNA-guided nuclease or RNA-guided DNA nuclease and a guide RNA, and optionally a donor template polynucleotide; a sequence-specific endonuclease and a donor template polynucleotide; one or more polynucleotides encoding an RNA-guided nuclease or RNA-guided DNA nuclease and a guide RNA, and optionally a donor template polynucleotide; one or more polynucleotide(s) encoding a sequence-specific endonuclease and a donor template polynucleotide; or any combination thereof; optionally such compositions further include at least one chemical, enzymatic, or physical delivery agent that provides for entry of the gene editing molecule into the plant cell.

Gene editing molecules for inducing a genetic modification in the plant cell or plant protoplast of the systems, methods, and compositions provided herein can include: (i) a polynucleotide selected from the group consisting of an RNA guide for an RNA-guided nuclease, a DNA encoding an RNA guide for an RNA-guided nuclease; (ii) a nuclease selected from the group consisting of an RNA-guided nuclease, an RNA-guided DNA endonuclease, a type II Cas nuclease, a Cas9, a type V Cas nuclease, a Cpf1, a CasY, a CasX, a C2c1, a C2c3, an engineered nuclease, a codon-optimized nuclease, a zinc-finger nuclease (ZFN), a transcription activator-like effector nuclease (TAL-effector nuclease), Argonaute, a meganuclease or engineered meganuclease; (iii) a polynucleotide encoding one or more nucleases capable of effecting site-specific modification of a target nucleotide sequence; and/or (iv) a donor template polynucleotide. In certain embodiments, the at least one delivery agent is selected from the group consisting of solvents, fluorocarbons, glycols or polyols, surfactants; primary, secondary, or tertiary amines and quaternary ammonium salts; organosilicone surfactants; lipids, lipoproteins, lipopolysaccharides; acids, bases, caustic agents; peptides, proteins, or enzymes; cell-penetrating peptides; RNase inhibitors; cationic branched or linear polymers; dendrimers; counter-ions, amines or polyamines, osmolytes, buffers, and salts; polynucleotides; transfection agents; antibiotics; chelating agents such as ammonium oxalate, EDTA, EGTA, or cyclohexane diamine tetraacetate, non-specific DNA double-strand-break-inducing agents; and antioxidants; particles or nanoparticles, magnetic particles or nanoparticles, abrasive or scarifying agents, needles or microneedles, matrices, and grids.

Gene editing molecules of use in the systems, methods, compositions, and reaction mixtures provided herein include molecules capable of introducing a double-strand break (“DSB”) in double-stranded DNA, such as in genomic DNA or in a target gene located within the genomic DNA as well as accompanying guide RNA or donor template polynucleotides. Examples of such gene editing molecules include: (a) a nuclease selected from the group consisting of an RNA-guided nuclease, an RNA-guided DNA endonuclease, a type II Cas nuclease, a Cas9, a type V Cas nuclease, a Cpf1, a CasY, a CasX, a C2c1, a C2c3, an engineered nuclease, a codon-optimized nuclease, a zinc-finger nuclease (ZFN), a transcription activator-like effector nuclease (TAL-effector nuclease), an Argonaute, and a meganuclease or engineered meganuclease; (b) a polynucleotide encoding one or more nucleases capable of effecting site-specific alteration (such as introduction of a DSB) of a target nucleotide sequence; (c) a guide RNA (gRNA) for an RNA-guided nuclease, or a DNA encoding a gRNA for an RNA-guided nuclease; and (d) donor template polynucleotides.

CRISPR (Clustered Regularly Interspaced Short Palindromic Repeats)/Cas (CRISPR-associated) systems, or CRISPR systems, are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. In a typical CRISPR/Cas system, a Cas endonuclease is directed to a target nucleotide sequence (e.g., a site in the genome that is to be sequence-edited) by sequence-specific, non-coding “guide RNAs” that target single- or double-stranded DNA sequences. In microbial hosts, CRISPR loci encode both Cas endonucleases and “CRISPR arrays” of the non-coding RNA elements that determine the specificity of the CRISPR-mediated nucleic acid cleavage.

Three classes (I-III) of CRISPR systems have been identified across a wide range of bacterial hosts and can be adapted for use in the systems, methods, and compositions provided herein. The well characterized class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically a 20-nucleotide RNA sequence that corresponds to (i.e., is identical or nearly identical to, or alternatively is complementary or nearly complementary to) a 20-nucleotide target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence.

The target DNA sequence must generally be adjacent to a “protospacer adjacent motif” (“PAM”) that is specific for a given Cas endonuclease; however, PAM sequences are short and relatively non-specific, appearing throughout a given genome. CRISPR endonucleases identified from various prokaryotic species have unique PAM sequence requirements. Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site.

Another class II CRISPR system can be adapted for use in the systems, methods, and compositions provided herein includes the type V endonuclease Cpf1 (also known as “Cas12a”), which is a smaller endonuclease than is Cas9; examples include AsCpf1 (from Acidaminococcus sp.) and LbCpf1 (from Lachnospiraceae sp.). In contrast to Cas9 type CRISPR systems, Cas12a-associated (“Cpf1”-associated) CRISPR arrays have been reported to be processed into mature crRNAs without the requirement of a tracrRNA, i.e., the naturally occurring Cas12a (Cpf1) CRISPR system was reported to require only the Cas12a (Cpf1) nuclease and a Cas12a crRNA to cleave the target DNA sequence; see Zetsche et al. (2015) Cell, 163:759-771; U.S. Pat. No. 9,790,490. Cpf1 endonucleases, are associated with T-rich PAM sites, e.g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771. Other CRISPR nucleases useful in methods, systems, and compositions disclosed herein include C2c1 and C2c3 (see Shmakov et al. (2015) Mol. Cell, 60:385-397). Like other CRISPR nucleases, C2c1 from Alicyclobacillus acidoterrestris (AacC2c1; amino acid sequence with accession ID TOD7A2, deposited on-line at www [dot] ncbi [dot] nlm [dot] nih [dot] gov/protein/1076761101) requires a guide RNA and PAM recognition site; C2c1 cleavage results in a staggered seven-nucleotide DSB in the target DNA (see Yang et al. (2016) Cell, 167:1814-1828.e12) and is reported to have high mismatch sensitivity, thus reducing off-target effects (see Liu et al. (2016) Mol. Cell, available on line at dx [dot] doi [dot] org/10 [dot] 1016/j [dot] molcel [dot] 2016 [dot] 11.040). Another CRISPR nuclease, Campylobacter jejuni-derived Cas9 (CjCas9), is only 984 amino acids in length (considerably smaller than, for example, S. pyogenes Cas9 at 1368 amino acids or S. aureus Cas9 at 1053 amino acids); CjCas9 also requires a guide RNA (reported to be optimal for a 22-nucleotide target sequence) and a PAM recognition site (reported to be 5′-NNNNACAC, 5′-NNNNRY, 5′-NNNNACA, or 5′-NNNNRYAC, where R is a purine and Y is a pyrimidine); see Kim et al. (2017) Nature Communications, 8:14500 (doi: 10.1038/ncomms14500). Yet other CRISPR nucleases include nucleases identified from the genomes of uncultivated microbes, such as CasX and CasY (e.g., a CRISPR-associated protein CasY from an uncultured Parcubacteria group bacterium, amino acid sequence with accession ID APG80656, deposited on-line at www [dot] ncbi [dot] nlm [dot] nih [dot] gov/protein/APG80656.1]); see Burstein et al. (2016) Nature, doi: 10.1038/nature21059.

CRISPR-type genome editing can be adapted for use in the systems, methods, and compositions provided herein in several ways. CRISPR elements, i.e., gene editing molecules comprising CRISPR endonucleases and CRISPR single-guide RNAs or polynucleotides encoding the same, are useful in effecting genome editing without remnants of the CRISPR elements or selective genetic markers occurring in progeny. In certain embodiments, the CRISPR elements are provided directly to the systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate. In certain embodiments, genome-inserted CRISPR elements are useful in plant lines adapted for use in the systems, methods, and compositions provide herein. In certain embodiments, plants or plant cells used in the systems, methods, and compositions provided herein can comprise a transgene that expresses a CRISPR endonuclease (e.g., a Cas9, a Cpf1-type or other CRISPR endonuclease). In certain embodiments, one or more CRISPR endonucleases with unique PAM recognition sites can be used. Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites. Introduction of one or more of a wide variety of CRISPR guide RNAs that interact with CRISPR endonucleases integrated into a plant genome or otherwise provided to a plant is useful for genetic editing for providing desired phenotypes or traits, for trait screening, or for trait introgression. Multiple endonucleases can be provided in expression cassettes with the appropriate promoters to allow multiple genome editing in a spatially or temporally separated fashion in either in chromosome DNA or episome DNA.

Whereas wild-type Cas9 generates double-strand breaks (DSBs) at specific DNA sequences targeted by a gRNA, a number of CRISPR endonucleases having modified functionalities can be used in the systems, methods, and compositions provided herein, for example: (1) a “nickase” version of Cas9 generates only a single-strand break; (2) a catalytically inactive Cas9 (“dCas9”) does not cut the target DNA but interferes with transcription; dCas9 can further be fused with a repressor peptide; (3) a catalytically inactive Cas9 (“dCas9”) fused to an activator peptide can activate or increase gene expression; (4) a catalytically inactive Cas9 (dCas9) fused to FokI nuclease (“dCas9-FokI”) can be used to generate DSBs at target sequences homologous to two gRNAs. See, e.g., the numerous CRISPR/Cas9 plasmids disclosed in and publicly available from the Addgene repository (Addgene, 75 Sidney St., Suite 550A, Cambridge, Mass. 02139; addgene [dot] org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Other CRISPR nucleases useful for editing genomes include C2c1 and C2c3 (see Shmakov et al. (2015) Mol. Cell, 60:385-397) and CasX and CasY (see Burstein et al. (2016) Nature, doi: 10.1038/nature21059). Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). All of the patent publications referenced in this paragraph are incorporated herein by reference in their entirety.

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least 16 or 17 nucleotides of gRNA sequence are required by Cas9 for DNA cleavage to occur; for Cpf1 at least 16 nucleotides of gRNA sequence are needed to achieve detectable DNA cleavage and at least 18 nucleotides of gRNA sequence were reported necessary for efficient DNA cleavage in vitro; see Zetsche et al. (2015) Cell, 163:759-771. In practice, guide RNA sequences are generally designed to have a length of 17-24 nucleotides (frequently 19, 20, or 21 nucleotides) and exact complementarity (i.e., perfect base-pairing) to the targeted gene or nucleic acid sequence; guide RNAs having less than 100% complementarity to the target sequence can be used (e.g., a gRNA with a length of 20 nucleotides and 1-4 mismatches to the target sequence) but can increase the potential for off-target effects. The design of effective guide RNAs for use in plant genome editing is disclosed in US Patent Application Publication 2015/0082478 A1, the entire specification of which is incorporated herein by reference. More recently, efficient gene editing has been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in genome editing; see, for example, Hendel et al. (2015) Nature Biotechnol., 985-991.

In certain embodiments, the guide RNA (gRNA) has a sequence of 16-24 nucleotides in length (e.g., 16, 17, 18, 19, 20, 21, 22, 23, or 24 nucleotides in length). Specific embodiments include gRNAs of 19, 20, or 21 nucleotides in length and having 100% complementarity to the target nucleotide sequence. In many embodiments the gRNA has exact complementarity (i.e., perfect base-pairing) to the target nucleotide sequence; in certain other embodiments the gRNA has less than 100% complementarity to the target nucleotide sequence. The design of effective gRNAs for use in plant genome editing is disclosed in US Patent Application Publication 2015/0082478 A1, the entire specification of which is incorporated herein by reference. In certain embodiments where multiple gRNAs are employed, the multiple gRNAs can be delivered separately (as separate RNA molecules or encoded by separate DNA molecules) or in combination, e.g., as an RNA molecule containing multiple gRNA sequences or as a DNA molecule encoding an RNA molecule containing multiple gRNA sequences; see, for example, US Patent Application Publication 2016/0264981 A1, the entire specification of which is incorporated herein by reference, which discloses RNA molecules including multiple RNA sequences (such as gRNA sequences) separated by tRNA cleavage sequences. Efficient Cas9-mediated gene editing has been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing).

Thus, in certain embodiments wherein the nuclease is a Cas9-type nuclease, the gRNA can be provided as a polynucleotide composition including: (a) a CRISPR RNA (crRNA) that includes the gRNA together with a separate tracrRNA, or (b) at least one polynucleotide that encodes a crRNA and a tracrRNA (on a single polynucleotide or on separate polynucleotides), or (c) at least one polynucleotide that is processed into one or more crRNAs and a tracrRNA. In other embodiments wherein the nuclease is a Cas9-type nuclease, the gRNA can be provided as a polynucleotide composition including a CRISPR RNA (crRNA) that includes the gRNA, and the required tracrRNA is provided in a separate composition or in a separate step, or is otherwise provided to the cell (for example, to a plant cell or plant protoplast that stably or transiently expresses the tracrRNA from a polynucleotide encoding the tracrRNA). In other embodiments wherein the nuclease is a Cas9-type nuclease, the gRNA can be provided as a polynucleotide composition comprising: (a) a single guide RNA (sgRNA) that includes the gRNA, or (b) a polynucleotide that encodes a sgRNA, or (c) a polynucleotide that is processed into a sgRNA. Cpf1-mediated gene editing does not require a tracrRNA; thus, in embodiments wherein the nuclease is a Cpf1-type nuclease, the gRNA is provided as a polynucleotide composition comprising (a) a CRISPR RNA (crRNA) that includes the gRNA, or (b) a polynucleotide that encodes a crRNA, or (c) a polynucleotide that is processed into a crRNA. In certain embodiments, the gRNA-containing composition optionally includes an RNA-guided nuclease, or a polynucleotide that encodes the RNA-guided nuclease. In other embodiments, an RNA-guided nuclease or a polynucleotide that encodes the RNA-guided nuclease is provided in a separate step. In some embodiments of the method, a gRNA is provided to a cell (e.g., a plant cell or plant protoplast) that includes an RNA-guided nuclease or a polynucleotide that encodes an RNA-guided nuclease, e g., an RNA-guided nuclease selected from the group consisting of an RNA-guided DNA endonuclease, a type II Cas nuclease, a Cas9, a type V Cas nuclease, a Cpf1, a CasY, a CasX, a C2c1, a C2c3, an engineered RNA-guided nuclease, and a codon-optimized RNA-guided nuclease; in an example, the cell (e.g., a plant cell or plant protoplast) stably or transiently expresses the RNA-guided nuclease. In certain embodiments, the polynucleotide that encodes the RNA-guided nuclease is, for example, DNA that encodes the RNA-guided nuclease and is stably integrated in the genome of a plant cell or plant protoplast, DNA or RNA that encodes the RNA-guided nuclease and is transiently present in or introduced into a plant cell or plant protoplast; such DNA or RNA can be introduced, e.g., by using a vector such as a plasmid or viral vector or as an mRNA, or as vector. In certain embodiments, the RNA-guided nuclease is provided as a ribonucleoprotein (RNP) complex, e.g., a preassembled RNP that includes the RNA-guided nuclease complexed with a polynucleotide including the gRNA or encoding a gRNA, or a preassembled RNP that includes a polynucleotide that encodes the RNA-guided nuclease (and optionally encodes the gRNA, or is provided with a separate polynucleotide including the gRNA or encoding a gRNA), complexed with a protein. In certain embodiments, the RNA-guided nuclease is a fusion protein, i.e., wherein the RNA-guided nuclease (e.g., Cas9, Cpf1, CasY, CasX, C2c1, or C2c3) is covalently bound through a peptide bond to a cell-penetrating peptide, a nuclear localization signal peptide, a chloroplast transit peptide, or a mitochondrial targeting peptide; such fusion proteins are conveniently encoded in a single nucleotide sequence, optionally including codons for linking amino acids. In certain embodiments, the RNA-guided nuclease or a polynucleotide that encodes the RNA-guided nuclease is provided as a complex with a cell-penetrating peptide or other transfecting agent. In certain embodiments, the RNA-guided nuclease or a polynucleotide that encodes the RNA-guided nuclease is complexed with, or covalently or non-covalently bound to, a further element, e.g., a carrier molecule, an antibody, an antigen, a viral movement protein, a polymer, a detectable label (e.g., a moiety detectable by fluorescence, radioactivity, or enzymatic or immunochemical reaction), a quantum dot, or a particulate or nanoparticulate. In certain embodiments, the RNA-guided nuclease or a polynucleotide that encodes the RNA-guided nuclease is provided in a solution, or is provided in a liposome, micelle, emulsion, reverse emulsion, suspension, or other mixed-phase composition. In certain embodiments, the DNA or RNA introduced directly into a plant cell or plant protoplast.

An RNA-guided nuclease can be provided to a cell (e.g., a plant cell or plant protoplast) by any suitable technique. In certain embodiments, the RNA-guided nuclease is provided by directly contacting a plant cell or plant protoplast with the RNA-guided nuclease or the polynucleotide that encodes the RNA-guided nuclease. In certain embodiments, the RNA-guided nuclease is provided by transporting the RNA-guided nuclease or a polynucleotide that encodes the RNA-guided nuclease into a plant cell or plant protoplast using a chemical, enzymatic, or physical agent as provided in detail in the paragraphs following the heading “Delivery Methods and Delivery Agents”. In certain embodiments, the RNA-guided nuclease is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of a plant cell or plant protoplast with a polynucleotide encoding the RNA-guided nuclease; see, e.g., Broothaerts et al. (2005) Nature, 433:629-633. In an embodiment, the RNA-guided nuclease is provided by transcription in a plant cell or plant protoplast of a DNA that encodes the RNA-guided nuclease and is stably integrated in the genome of the plant cell or plant protoplast or that is provided to the plant cell or plant protoplast in the form of a plasmid or expression vector (e.g., a viral vector) that encodes the RNA-guided nuclease (and optionally encodes one or more gRNAs, crRNAs, or sgRNAs, or is optionally provided with a separate plasmid or vector that encodes one or more gRNAs, crRNAs, or sgRNAs). In certain embodiments, the RNA-guided nuclease is provided to the plant cell or plant protoplast as a polynucleotide that encodes the RNA-guided nuclease, e.g., in the form of an mRNA encoding the nuclease.

Where a polynucleotide is concerned (e.g., a crRNA that includes the gRNA together with a separate tracrRNA, or a crRNA and a tracrRNA encoded on a single polynucleotide or on separate polynucleotides, or at least one polynucleotide that is processed into one or more crRNAs and a tracrRNA, or a sgRNA that includes the gRNA, or a polynucleotide that encodes a sgRNA, or a polynucleotide that is processed into a sgRNA, or a polynucleotide that encodes the RNA-guided nuclease), embodiments of the polynucleotide include: (a) double-stranded RNA; (b) single-stranded RNA; (c) chemically modified RNA; (d) double-stranded DNA; (e) single-stranded DNA; (f) chemically modified DNA; or (g) a combination of (a)-(f). Where expression of a polynucleotide is involved (e.g., expression of a crRNA from a DNA encoding the crRNA, or expression and translation of a RNA-guided nuclease from a DNA encoding the nuclease), in some embodiments it is sufficient that expression be transient, i.e., not necessarily permanent or stable in the cell. Certain embodiments of the polynucleotide further include additional nucleotide sequences that provide useful functionality; non-limiting examples of such additional nucleotide sequences include an aptamer or riboswitch sequence, nucleotide sequence that provides secondary structure such as stem-loops or that provides a sequence-specific site for an enzyme (e.g., a sequence-specific recombinase or endonuclease site), T-DNA (e.g., DNA sequence encoding a gRNA, crRNA, tracrRNA, or sgRNA is enclosed between left and right T-DNA borders from Agrobacterium spp, or from other bacteria that infect or induce tumours in plants), a DNA nuclear-targeting sequence, a regulatory sequence such as a promoter sequence, and a transcript-stabilizing sequence. Certain embodiments of the polynucleotide include those wherein the polynucleotide is complexed with, or covalently or non-covalently bound to, a non-nucleic acid element, e.g., a carrier molecule, an antibody, an antigen, a viral movement protein, a cell-penetrating or pore-forming peptide, a polymer, a detectable label, a quantum dot, or a particulate or nanoparticulate.

Other nucleases capable of effecting site-specific (sequence specific) modification of a target nucleotide sequence in the systems, methods, and compositions provided herein include zinc-finger nucleases (ZFNs), transcription activator-like effector nucleases (TAL-effector nucleases or TALENs), Argonaute proteins, and a meganuclease or engineered meganuclease. Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see, e.g., Urnov et al. (2010) Nature Rev. Genet., 11:636-646. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotides bases outside their target triplet. Typically, the engineered zinc finger DNA binding domain has a novel binding specificity, compared to a naturally-occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) are well known and described in the literature. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as FokI. This endonuclease must dimerize to cleave DNA. Thus, cleavage by FokI as part of a ZFN requires two adjacent and independent binding events, which must occur in both the correct orientation and with appropriate spacing to permit dimer formation. The requirement for two DNA binding events enables more specific targeting of long and potentially unique recognition sites. FokI variants with enhanced activities have been described; see, e.g., Guo et al. (2010) J. Mol. Biol., 400:96-107.

### Regeneration and Seed Production

Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petty dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to have the described transgene. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants to introgress the transgene into the genome of the inbred plants. This process is referred to as backcross conversion. When a sufficient number of crosses to the recurrent inbred parent have been completed to produce a product of the backcross conversion process that is substantially isogenic with the recurrent inbred parent except for the presence of the introduced expression cassette encoding a TGNap1 gene, the plant can be self-pollinated at least once to produce a homozygous backcross converted inbred containing the transgene or expression cassette. Progeny of these plants are true breeding.

Alternatively, seed from transformed plant lines regenerated from transformed tissue cultures can be grown in the field and self-pollinated to generate true breeding plants.

Seed from the fertile transgenic plants can then be evaluated for the presence of the desired TGNap1 genomic modification, e.g., the desired TGNap1 expression cassette, and/or the expression of the desired TGNap1 protein. Transgenic plant and/or seed tissue can be analyzed using standard methods such as SDS polyacrylamide gel electrophoresis, liquid chromatography (e.g., HPLC) or other means of detecting a mutation.

Once a transgenic plant with a mutant sequence and having wild type growth and pathogen resistance is identified, seeds from such plants can be used to develop true breeding plants. The true breeding plants are used to develop a line of plants with improved growth and pathogen resistance relative to wild type, and acceptable bacterial pathogen resistance while still maintaining other desirable functional agronomic traits. Adding the mutation to other plants can be accomplished by hack-crossing with this trait and with plants that do not exhibit this trait and studying the pattern of inheritance in segregating generations. Those plants expressing the target trait (e.g., TGNap1 overexpression, good growth) in a dominant fashion are preferably selected. Back-crossing is carried out by crossing the original fertile transgenic plants with a plant from an inbred line exhibiting desirable functional agronomic characteristics while not necessarily expressing the trait of increased herbicide and pathogen resistance and good plant growth. The resulting progeny are then crossed back to the parent that expresses the increased herbicide and pathogen resistance and good plant growth. The progeny from this cross will also segregate so that some of the progeny carry the trait and some do not. This back-crossing is repeated until an inbred line with the desirable functional agronomic traits, and with expression of the trait involving an increase in herbicide and pathogen resistance and good plant growth. Such herbicide and pathogen resistance as well as good plant growth can be expressed in a dominant fashion.

The new transgenic plants can also be evaluated for a battery of functional agronomic characteristics such as growth, lodging, kernel hardness, yield, resistance to disease and insect pests, drought resistance, and/or herbicide resistance.

### Determination of Stably Transformed Plant Tissues

To confirm the presence of TGNap1 expression cassette in the regenerating plants, or seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced TGNap1 transgene or expression cassette. For example, PCR also be used to reverse transcribe RNA into DNA, using enzymes such as reverse transcriptase, and then this DNA can be amplified through PCR techniques.

Further information about the nature of the RNA product may be obtained by Northern blotting. This technique will demonstrate the presence of an RNA species and give information about the integrity of that RNA. The presence or absence of an RNA species (e.g., TGNap1 RNA) can also be determined using dot or slot blot Northern hybridizations. These techniques are modifications of Northern blotting and also demonstrate the presence or absence of an RNA species.

While Southern blotting and PCR may used to detect the presence of a TGNap1 expression cassette, they do not provide information as to whether the preselected DNA segment is being expressed. Expression may be evaluated by specifically identifying the protein products of the introduced TGNap1 expression cassette or the introduced mutations, by detecting expression of TGNap1 proteins, or evaluating the phenotypic changes brought about by such mutation.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products, or the absence thereof, that have been separated by electrophoretic techniques. Additional techniques may be employed to absolutely confirm the identity of a mutation such as evaluation by screening for reduced transcription (or no transcription) of TGNap1 mRNAs, by screening for the TGNap1 mRNA or TGNap1 protein expression. Amino acid sequencing following purification can also be employed. The Examples of this application also provide assay procedures for detecting and quantifying infection and plant growth. Other procedures may be additionally used.

The expression of a gene product can also be determined by evaluating the phenotypic results of its expression. These assays also may take many forms including but not limited to analyzing changes growth or growth characteristics (e.g., taller plants), detection of stronger fibers, observation of greater mechanical strength, or other physiological properties of the plant. Expression of selected DNA segments encoding different amino acids or having different sequences and may be detected by amino acid analysis or sequencing.

### EXAMPLES

### Example 1: TGNap1 Provides for a Robust Immune Response

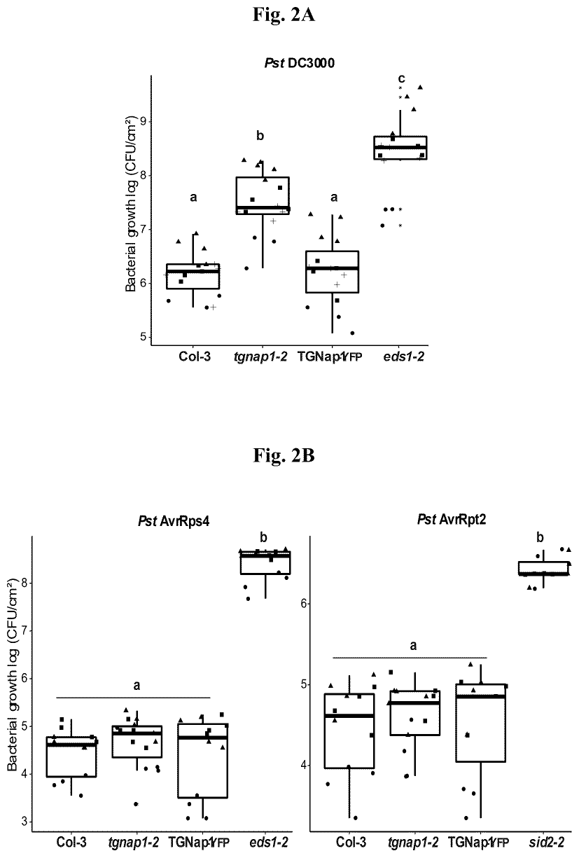

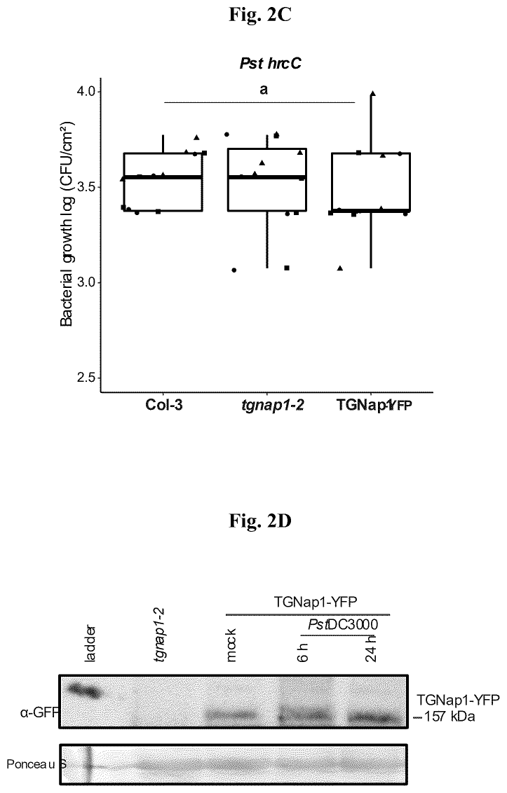

We adopted TGNap1 as a tool to gain insights into TGN-dependent mechanisms for the traffic of antimicrobial proteins in plant immunity. We infected a complete TGNap1 knockout (tgnap1-2) with the virulent pathogen Pst DC3000. Compared to Col-3 (wild-type; WT), we found that tgnap1-2 was more susceptible to infection by Pst DC3000 than WT, displaying eight-tenfold increased bacterial growth but less than the hypersusceptible mutant enhanced disease susceptibility 1 (eds1-2) (FIGS. 2A-2C). To test if different ecotypes could account for the increased bacterial growth, we infected Col-0 and Col-3 plants with Pst DC3000 and observed that both Col-0 and Col-3 plants had comparable susceptibility, while gnap 1-2 and eds1-2 (in Col-0) were hypersusceptible. Complementing tgnap1-2 with a full-length TGNap1 fused to YFP (TGNap1-YFP) restored the susceptibility of tgnap1-2 to WT levels (FIG. 2A). Thus, TGNap1 contributes to plant immunity.

Because we found that tgnap1-2 is hypersusceptible to Pst DC3000 (FIG. 2A), we next tested the involvement of TGNap1 in ETI and PTI. To do so, we infiltrated WT and tgnap1-2 plants with Pst AvrRps4 and Pst AvrRpt2, which are recognized by the products of Toll-Interleukin-1 receptor domain-NLR gene pair, RPS4 (Resistance to Pseudomonas syringae 4) and RRS/(resistance to Ralstonia solanacearum 1), and of the coiled-coil NLR RPS2 (Resistance to Pseudomonas syringae 2), respectively (FIG. 2B). Both tgnap1-2 and TGNap1-YFP displayed WT-like resistance, while eds1-2 and the SA-deficient mutant, sid2-2, were susceptible against Pst AvrRps4 (TNL) and Pst AvrRpt2 (CNL), respectively (FIG. 2B). These results indicate that, although tgnap1-2 has a significant role in basal immunity against Pst DC3000, it is dispensable during ETI. Next, to assess a role of TGNap1 in PTI, we used flg22, a 22-amino acid peptide of flagellin, which activates PTI. We infiltrated flg22 into Col, Ignap1-2, TGNap1-YFP, and fec (a triple mutant of PRR receptors fls2, efr, and cerk1 (FLS2-Flagellin sensing 2; EFR-Elongation factor-Tu receptor; CERK1-Chitin elicitor receptor kinase 1). Recognition of flg22 by FLS2 leads to rapid activation of mitogen-activated proteins kinase (MPK) signaling cascades. Upon flg22 treatment, MPK3/MPK6 phosphorylation in tgnap1-2 was indistinguishable from Col and TGNap1-YFP, indicating that PTI-activated MPK signaling cascades are not affected by the loss of TGNap1. In addition, we tested if tgnap1-2 was required in resistance to PTI-activating Pst hrcC, a Pst strain with a defective Type-III-secretion system. Upon infection with Pst hrcC, the bacterial growth in tgnap1-2 was comparable to Col-3 and TGNap1-YFP (FIG. 2C). The observed WT-like levels of bacterial growth of tgnap1-2 to Pst hrcC is suggestive of one or more Pst-elicited effectors targeting TGNap1 functions or of the possibility that TGNap1 is recruited specifically in basal defense against Pst DC3000. To test if the immune role of TGNap1 was limited to Pst DC3000, we checked if tgnap1-2 was susceptible to the Oomycete Hyaloperonospora arabidopsidis (Hpa) NOCO2, which causes downy mildew. Upon infection with Hpa NOCO2, the tgnap1-2 mutant showed intermediate susceptibility between Col-3 and the hypersusceptible eds1-2, while no spores were observed in the Hpa NOCO2-resistant Ws-2 plants. Collectively, these results indicate a functional role of TGNap1 in limiting virulent Pst DC3000 and Hpa, but not in ETI or Pst hrcC-mediated PTI.

Next, we aimed to gain insights on whether the observed hyper-susceptibility of tgnap1-2 to Pst DC3000 could be due to a specific pathogen-elicited effector. Pst DC3000 contains 36 effectors, of which 8 are sufficient to restore much of the Pst virulence in Nicotiana benthamiana. We infected Col-3 and tgnap1-2 plants with the following Pst mutants: Pst avrPto avrPtoB (encoding effectors involved in immune suppression), Pst avrE hopMI (ΔEM) (encoding effectors involved in water-soaking and suppression of PTI), and Pst ΔCEL (a deletion mutant of the conserved effector locus, including avrE and hopMI, leading to reduced virulence) S. Compared to Pst DC3000, both Pst avrPto avrPtoB and Pst ΔEM grew less on Col-3, underscoring their roles in virulence of Pst DC3000 in this ecotype (FIG. 2A). However, tgnap1-2 showed increased susceptibility, with 5-6 fold higher bacterial growth compared to Col-3 against both Pst avrPto avrPtoB and Pst ΔEM. Against Pst ΔCEL, tgnap1-2 showed a significant but less pronounced increase in bacterial load compared to Col-3, supporting that, with reduced virulence, the pathogen growth differenTGNap1 between Col-3 and tgnap1-2 were also reduced, but not absent. TGNap1 transcript and protein levels were unaltered upon Pst DC3000 infiltration (FIG. 2D). Taken together, these results underscore the role of TGNap1 in basal immunity, which is likely separate from canonical ETI and PTI defense pathways targeted by Pst effectors.

### Example 2: TGNap1 is Involved in the Secretion of Antimicrobial Proteins

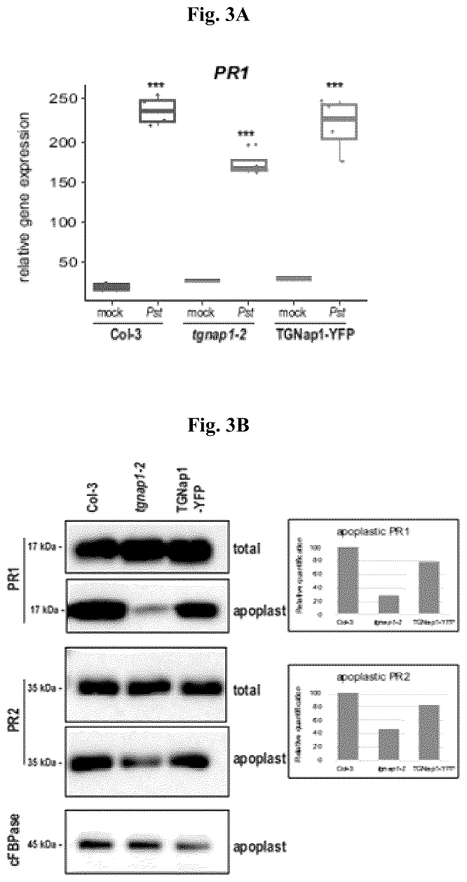

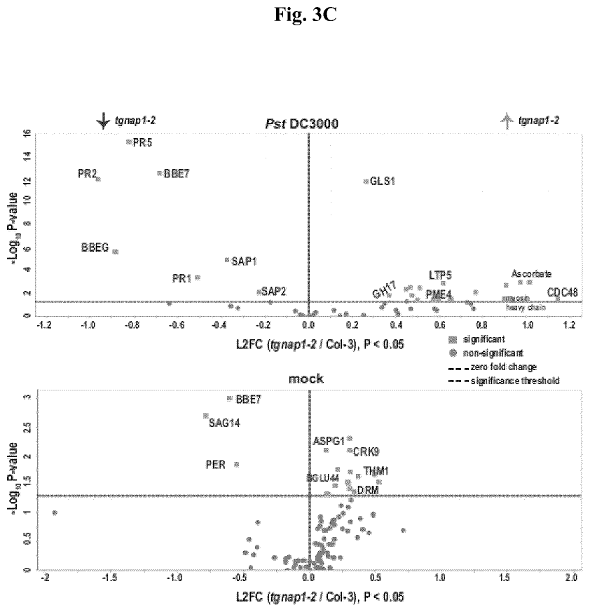

We hypothesized that TGNap1 could play a role in trafficking of antimicrobial proteins to the apoplast. To test this, we aimed to determine the transcript and protein levels of the antimicrobial protein, PR1, upon Pst DC3000 treatment. We found that, upon Pst DC3000 treatment, PR1 transcript levels were induced in Col-3, tgnap1-2 and TGNap1-YFP infected leaves (FIG. 3A). The total protein levels of PR1 and PR2 (FIG. 3B) were comparable between Col-3, tgnap1-2 and TGNap1-YFP infected leaves, indicating that the loss of TGNap1 does not affect the abundance of PR1 and PR2. This is in agreement with canonical SA-mediated defense pathways not being affected in tgnap1-2. Next, to test whether PR1 and PR2 secretion into the apoplast was affected by the loss of TGNap1, we isolated apoplastic fluids from leaves infiltrated with either mock or Pst DC3000. Total protein and apoplastic protein fractions were probed with α-PR1 and α-PR2 specific antibodies. Col-3, tgnap1-2, and TGNap1-YFP accumulated comparable levels of total PR1 and PR2 protein, but notably, only tgnap1-2 displayed reduced PR1 levels in the apoplastic fraction (FIG. 3B). To determine the levels of cytosolic protein contamination during apoplast fluid isolation, the protein samples were probed with antibodies to the cytosolic marker cytosolic fructose-1,6-bisphosphatase (cFBPase). We detected some cFBPase signal at comparable levels across all samples (FIG. 3B), indicating that the differenTGNap1 in the levels of PR1 and PR2 between Col and tgnap1-2 were not due to cytosolic contamination. Indeed, higher levels of PR1 were found in the intracellular extracts from tgnap1-2 compared to Col-3 (intracellular). These results show that TGNap1 is involved in the secretion of PR1 and PR2 to the apoplast upon immune activation.

To test if other proteins were transported to the apoplast in a TGNap1-dependent pathway during immunity, we quantitatively and qualitatively assayed and compared the apoplastic fluid proteome of Col-3 and tgnap1-2 before and after immune activation. To do this, we isolated apoplastic fluids from Arabidopsis leaves infiltrated with either mock or Pst DC3000 and subjected it to isobaric peptide labeling (labeling efficiency: mock 94%, and Pst DC3000-infiltrated samples 100%), for protein identification by MS/MS. The plant genotypes were paired for comparison based on treatment (i.e., mock or Pst DC3000, to account for differenTGNap1 in protein population and abundance). A total of 940 and 922 proteins were detected in mock and Pst DC3000 samples, respectively. From the total detected proteins, a cutoff (minimum of two peptides and 1% FDR) was set to identify differentially abundant proteins. 793 and 786 proteins were detected with this cutoff in the apoplast fluids of mock and Pst DC3000 samples, respectively. A significant overlap was observed in the apoplastic proteome of mock and Pst DC3000-treated Col-3 and tgnap1-2 plants. A GO-enrichment analysis of proteins found in both mock and Pst DC3000-treated genotypes revealed a significant representation of proteins involved in stress responses (e.g., pathogenesis-related proteins, aspartyl proteases, pectin methylesterases, lipid-transfer proteins (LTP5)). The data were further subjected to permutation test (P<0.05 post-hoc: Benjamini-Hochberg) to account for variation between samples and multiple-testing errors. Using a criterion of +1 fold change, we found that, compared to Col-3, 18 proteins were more abundant in tgnap1-2 and 7 proteins were less abundant in tgnap1-2 in Pst DC3000-treated plants compared to Col-3. The 18 proteins more abundant in tgnap1-2 relative to Col-3, included proteins involved in biotic stress (e.g., pectin methyl esterase 4 (PME4), lipid-transfer protein 5 (LTP5), glycine-rich protein 3 and glycine-rich protein 3 short isoform) and abiotic stress (e.g., ascorbate oxidase, responsive to desiccation 22, glycosyl hydrolase 17 (GH17)) (FIG. 3C). Interestingly, upon Pst DC3000 infection, the proteins less abundant in tgnap1-2 apoplast compared to Col-3 were exclusively represented by antimicrobial proteins (FIG. 3C) of the pathogenesis-related proteins family (i.e., PR1, PR2, PR5), berberine-bridge enzymes (BBE4, BBE7), and secreted serine aspartic proteases (SAP1 and SAP2). Except BBE7, none of the above proteins were differentially abundant in the apoplast of mock-treated Col-3 and tgnap1-2 plants. These results are consistent with role of TGNap1 in trafficking of apoplastic proteins but also highlight that TGNap1 is required for efficient secretion of antimicrobial proteins upon Pst DC3000 infection. Taken together, our results show that during Pst DC3000 infection the loss of TGNap1 does not affect immune activation but plays a significant role in the transport of anti-bacterial proteins to the apoplast.

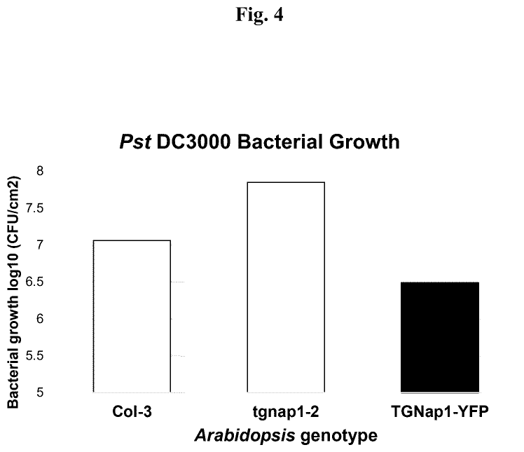

Arabidopsis plants of the indicated genotypes: wild type (Col-3), complete TGNap1 knockout (tgnap1-2), and TGNap1-YFP transgenic plants were infiltrated with Pst DC3000 and bacterial growth was measured (FIG. 4). Reduced bacterial growth in the TGNap1-YFP transgenic plants was observed relative to the TGNap1 knockout (tgnap1-2) plants.

### Example 3: TGNap1 Provides for a Robust Immune Response in Tomato Plants

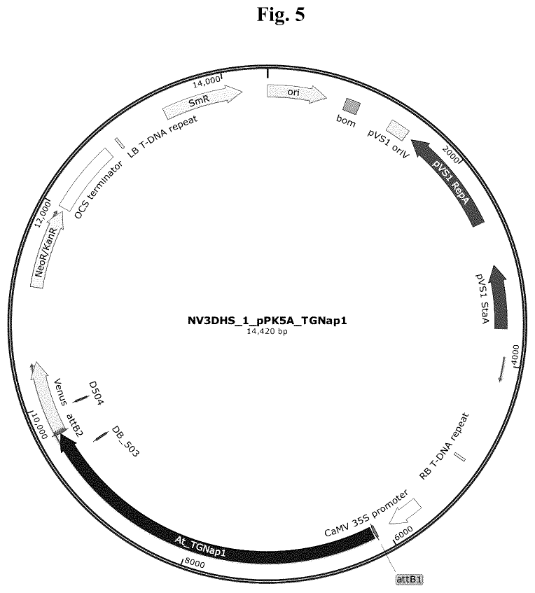

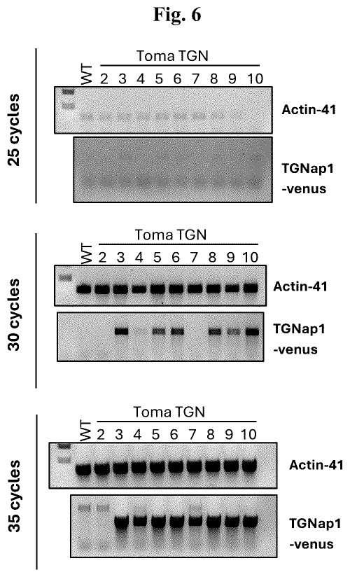

Tomato transgenic lines were created using a vector with an Arabidopsis TGNap1 Venus fusion (TGNap1-Venus) gene driven by a 35s promoter (FIG. 5). The resulting transgenic lines were characterized using RT-PCR analysis (25, 30, and 35 PCR cycles) of TGNap1-Venus, Actin-41 was used as a house-keeping gene. The TomTGN #2 line with no detectable TGNap1 level was not analyzed further but used as an internal control (FIG. 6).

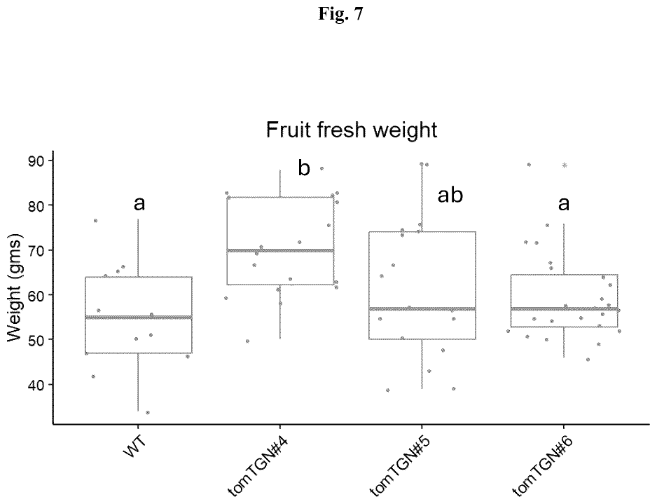

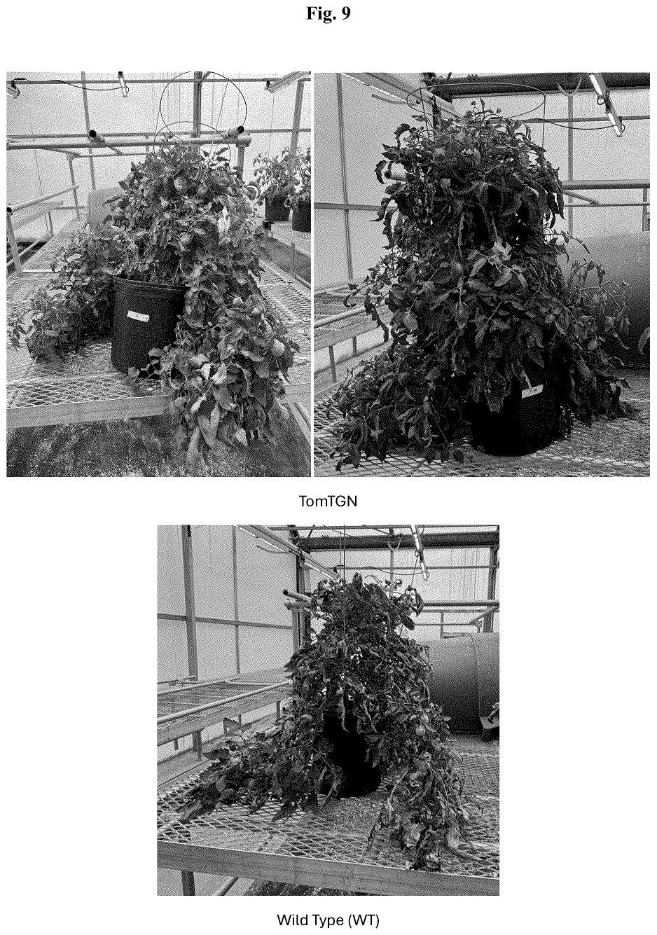

Plants were further characterized to determine if there were any changes in yield or growth. Fruit fresh from tomato transgenic lines expressing Arabidopsis TGNap1 and wild type tomatoes were weighed (FIG. 7). The difference in fresh fruit weight between lines was measured using Anova (Tukey HSD α<0.05). There was no decrease in fresh fruit weight in Tomato transgenic lines expressing Arabidopsis TGNap1. Tomato transgenic lines expressing Arabidopsis TGNap1 and wild type tomato lines were grown and compared (FIG. 9). There was no significant change in plant growth between transgenic lines expressing Arabidopsis TGNap1 and wild type tomato lines.

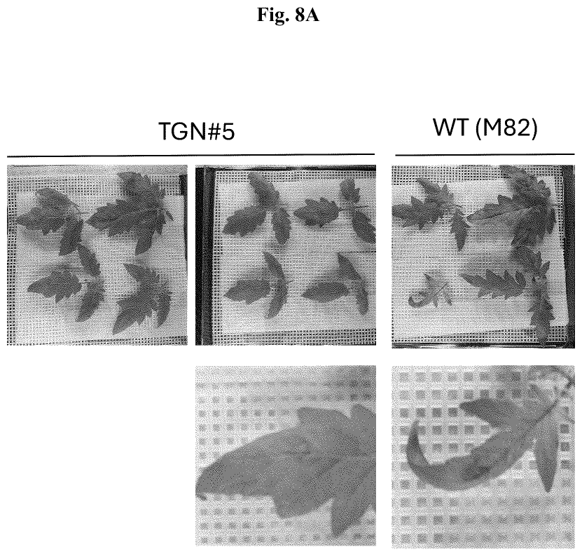

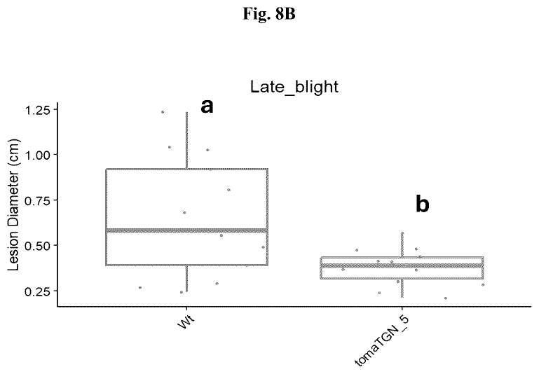

Plant resistance to late blight was tested. Leaves from tomato transgenic lines expressing Arabidopsis TGNap1 (tomTGN #5) and wild type tomato lines (WT) were infected with Phytophthora infectants. Images of leaves with late blight lesions were taken at 5 dpi and quantified (FIG. 8A). The difference in lesion diameter between genotypes was measured using Anova (Tukey HSD α<0.05) (FIG. 8B). There was a significant decrease in lesion diameter in tomato transgenic lines expressing Arabidopsis TGNap1 when compared to wild type lines.

All cited patents and patent publications referred to in this application are incorporated herein by reference in their entirety. All of the materials and methods disclosed and claimed herein can be made and used without undue experimentation as instructed by the above disclosure and illustrated by the examples. Although the materials and methods of this disclosure have been described in terms of embodiments and illustrative examples, it will be apparent to those of skill in the art that substitutions and variations can be applied to the materials and methods described herein without departing from the concept, spirit, and scope of the disclosure. All such similar substitutes and modifications apparent to those skilled in the art are deemed to be within the spirit, scope, and concept of the disclosure as encompassed by the embodiments of the disclosures recited herein and the specification and appended claims.

What is claimed:

1. A transgenic plant, plant seed, or plant cell comprising an expression cassette comprising a promoter operably linked to a nucleic acid segment encoding a TGNap1 protein with at least 90% sequence identity to any of SEQ ID NO: 1-13 or an allelic variant thereof, wherein TGNap1 is overexpressed as compared to a control plant.

2. The transgenic plant, plant seed, or plant cell of claim 1, wherein the plant has wild type growth and/or wild type biomass as compared to the control plant.

3. The transgenic plant, plant seed, or plant cell of claim 1, wherein growth of a bacterial plant pathogen, powdery mildew pathogen, and/or an Oomycete pathogen on the transgenic plant is reduced by approximately five-fold as compared to the control plant.

4. The transgenic plant, plant seed, or plant cell of claim 1, wherein the bacterial plant pathogen comprises Pseudomonas syringae DC3000 or wherein the Oomycete pathogen comprises downy mildew.

5. A transgenic plant, plant seed, or plant cell comprising a heterologous promoter operably linked to a nucleic acid segment encoding an endogenous TGNap1 protein with at least 90% sequence identity to any of SEQ ID NO: 1-13 or an allelic variant thereof, wherein TGNap1 is overexpressed as compared to a control plant.

6. The transgenic plant, plant seed, or plant cell of claim 5, wherein the plant has wild type growth and/or wild type biomass as compared to the control plant.

7. The transgenic plant, plant seed, or plant cell of claim 5, wherein growth of a bacterial plant pathogen, powdery mildew pathogen, and/or an Oomycete pathogen on the transgenic plant is reduced by approximately five-fold as compared to the control plant.

8. The transgenic plant, plant seed, or plant cell of claim 5, wherein the bacterial plant pathogen comprises Pseudomonas syringae DC3000/or Oomycete pathogen comprises downy mildew.

9. The transgenic plant, plant seed, or plant cell of claim 5, wherein the nucleic acid segment encoding the endogenous TGNap1 protein comprises an endogenous TGNap1 gene coding region.

10. The transgenic plant, plant seed, or plant cell of claim 9, wherein the endogenous TGNap1 gene coding region is located at its native location in the plant genome.

11. The transgenic plant, plant seed, or plant cell of claim 10, wherein the heterologous promoter comprises an insertion of one or more transcriptional enhancers in the endogenous TGNap1 gene promoter located at its native location in the plant genome.

12. A biological sample obtained from the transgenic plant, plant seed, or plant cell of claim 5, wherein the biological sample comprises the heterologous promoter operably linked to the nucleic acid segment encoding the endogenous TGNap1 protein.

13. The biological sample of claim 12, wherein the biological sample comprises grain meal or a plant tissue homogenate.

14. A biological sample obtained from the transgenic plant, plant seed, or plant cell of claim 5, wherein the biological sample comprises the expression cassette.

15. A method comprising cultivating a seed or plant comprising an expression cassette comprising a promoter operably linked to a nucleic acid segment encoding a TGNap1 protein with at least 90% sequence identity to any of SEQ ID NO: 1-13 or an allelic variant thereof, wherein TGNap1 is overexpressed as compared to a control plant; and/or comprising a heterologous promoter operably linked to a nucleic acid segment encoding an endogenous TGNap1 protein with at least 90% sequence identity to any of SEQ ID NO: 1-13 or an allelic variant thereof, wherein TGNap1 is overexpressed as compared to a control plant.

16. The method of claim 15, wherein the seed or the plant is Arabidopsis thaliana, grape, cotton, soybean, tomato, potato, rice, sorghum, corn, coffee, wheat, citrus (e.g. orange), or an apple seed or plant.

17. The method of claim 17, further comprising harvesting cotton from the cotton plant or obtaining seed from the Arabidopsis thaliana, grape, cotton, soybean, tomato, potato, rice, sorghum, corn, coffee, wheat, citrus (e.g. orange), or apple plant.

18. The method of claim 15, wherein the plant has wild type growth and/or wild type biomass as compared to the control plant.

19. The method of claim 15, wherein growth of a bacterial plant pathogen, powdery mildew pathogen, and/or an oomycete pathogen on the transgenic plant is reduced by approximately five-fold as compared to the control plant.

20. The method of claim 19, wherein the bacterial plant pathogen comprises Pseudomonas syringae or the Oomycete pathogen comprises downy mildew.
